(19) United States
(12) Patent Application Publication
(10) Pub. No.: US 2016/0194599 A1
(43) Pub. Date: Jul. 7, 2016

(54) NOVEL METHOD FOR ADHERENT CULTURE IN REGION FORMED BETWEEN WATER-ABSORBENT POLYMER GEL AND SUBSTRATE, METHOD FOR MANUFACTURING BIOMASS, AND NOVEL MICROALGA

(71) Applicant: FUJIFILM Corporation, Tokyo (JP)

(72) Inventor: Hideyuki KANEHARA, Ashigarakami-gun (JP)

(73) Assignee: FUJIFILM Corporation, Tokyo (JP)

(21) Appl. No.: 15/074,846

(22) Filed: Mar. 18, 2016

Related U.S. Application Data
(63) Continuation of application No. PCT/JP2014/074958, filed on Sep. 19, 2014.

(30) Foreign Application Priority Data
Sep. 20, 2013 (JP) 2013-194973
Sep. 20, 2013 (JP) 2013-194974
Mar. 20, 2014 (JP) 2014-058126

(51) Int. Cl. C12N 1/12; C12P 7/64; C12N 1/16

(52) U.S. Cl. CPC C12N 1/12; C12N 1/16; C12P 7/64

(57) ABSTRACT

An object of the present invention is to provide a method in which it is possible to reduce costs of manufacturing biomass derived from microorganisms, in particular, costs of manufacturing biomass derived from microalgae. Microorganisms are cultured within a region surrounded by water-absorbent polymer gel and a substrate therebetween. The culturing can also be performed in a state in which the structure consisting of the water-absorbent gel and the substrate is horizontally installed on the ground, and in a state in which the structure thereof is installed on the ground at a certain angle. In addition, the culturing can also be performed in a state in which the structure thereof is vertically installed on the ground.

### CROSS-REFERENCE TO RELATED APPLICATIONS

This application is a Continuation of PCT International Application No. PCT/JP2014/074958 filed on Sep. 19, 2014, which claims priority under 35 U.S.C §119(a) to Japanese Patent Application No. 2013-194973 filed on Sep. 20, 2013, Japanese Patent Application No. 2013-194974 filed on Sep. 20, 2013 and Japanese Patent Application No. 2014-058126 filed on Mar. 20, 2014. Each of the above application(s) is hereby expressly incorporated by reference, in its entirety, into the present application.

### BACKGROUND OF THE INVENTION

1. Field of the Invention

The present invention relates to a method for culturing microorganisms in a region formed between a substrate and the top of a gel, which contains a medium capable of favorably culturing microorganisms and is formed of a water-absorbent polymer compound.

2. Description of the Related Art

Microorganisms cannot be substantially cultured under the condition in which there is no water, and therefore, means for supplying moisture through a certain method is required.

Culturing of microorganisms is performed using a large amount of water. A broad swath of land is required for producing biofuels using microalgae. It is possible to consider land such as a dessert with a small amount of rainfall if use of land such as agricultural land with a high additional value is to be avoided. However, in such a region, in many cases, it is difficult to secure water and to use a large amount of water. Furthermore, a large amount of energy is required for handling a large amount of water, and therefore, it is desired to efficiently use water.

A colony is formed by preparing a gel using an agar medium or the like, coating the surface of the gel with a solution containing unpurified microorganisms using a platinum loop or the like, and then, culturing the coated gel. Purification of microorganisms is performed by picking up the colony. That is, it is possible to make microorganisms proliferate on a water-absorbent polymer. This is because the water-absorbent polymer can accumulate a large amount of moisture within a molecule and supply moisture to microorganisms.

Taking this into consideration, the present inventors have reported that it is possible to culture photosynthetic microorganisms on a substrate after forming a water-absorbent polymer layer thereon (JP2012-157274A). In addition, a method for raising round green algae within a container filled with water-absorbent polymers that are swollen with water is disclosed (in JP2008-187970A).

In recent years, an increase in fuel prices which is thought to be caused by the use of a large amount of fossil fuels due to development in industrial activities, or the progression of global warming caused by the green house effect due to carbon dioxide being released into the air has become a problem. In order to solve such a problem, development for immobilizing carbon dioxide using light energy using microalgae, which have been reported to have high oil productivity per unit area, and for converting carbon dioxide into a hydrocarbon compound, biodiesel (triglyceride), or the like, has been attracting attention. However, this method is an expensive culture method in which it is difficult to lower the energy consumed for each of the processes, and therefore, production is not performed on a commercial scale, except for a part of biomass.

As a task to be examined while lowering the energy in the above-described processes, the amount of energy to be input to a drying process of collected microalgae, which is required for taking out fuels such as oil, has been considered a problem. In this process, the amount of energy to be input for drying microalgae becomes larger as the moisture content in a collected substance is higher, and therefore, it is focused on how the moisture content in a collected substance can be reduced (Mitsufumi MATSUMOTO, et al., The 62nd Annual Meeting of the Society for Biotechnology, Japan, (2010) Topics Collection, p. 33). For example, it is reported that, in Microcystis in which it is considered that algal bodies exist so as to be comparatively localized within a culture space, the algal bodies are distributed while the number of algal bodies gradually decreases from the surface of water into the water, and therefore, the collection rate is low being about 80% and the moisture content of concentrated Microcystis obtained through centrifugal separation is 97% in average (Ebara Engineering Review, p. 32, No. 217 (2007-10).

### SUMMARY OF THE INVENTION

The problem in production of biomass using microorganisms is that there has been no development in an efficient culture method, an efficient collection method, or an efficient extraction method of useful substances (such as oil), and costs are increased.

Culturing of microorganisms uses a large amount of water since the culturing is performed while dispersing microorganisms in a medium. For this reason, it is necessary to stir the medium by inputting a large amount of energy. An object of the present invention is to provide a method for efficiently culturing microorganisms using the minimum amount of water without performing stirring at all.

In general, the size of microorganism is small, and therefore, various methods such as collection through filtration, collection after making an aggregation of microorganisms using a precipitant, and collection using a centrifugal separator have been examined as methods for collecting microorganisms from a culture solution. However, there are various problems such as the occurrence of clogging, a large amount of energy to be input, and high costs. In order to improve such problems, another object of the present invention is to provide a method for efficiently collecting microorganisms.

As a high-density culture method, there is an adhesion culture method or a water surface-floating culture method. However, there is a significant problem in that these methods depend on the properties of microorganisms. Still another object of the present invention is basically to achieve high-density culture without depending on the types of microorganisms.

Furthermore, the oil extraction efficiency decreases as the moisture content in a collected substance is higher. For this reason, the oil extraction efficiency is improved by performing drying treatment. However, there is a problem in that a larger amount of energy for drying is required as the moisture content in a collected substance is higher. Therefore, still another problem of the present invention is to significantly reduce the moisture content in a collected substance which has been obtained through a collection operation in comparison with a conventional method.

In the conventional method, in many cases, a culture vessel is an open system in a case of performing culturing on a commercial scale, and therefore, there is a problem in that non-target microorganisms, or organisms that prey on microorganisms, that is, contamination microorganisms, enter a medium. Still another object of the present invention is to provide a method for suppressing the entrance of contamination microorganisms as much as possible.

In addition, in a case where the microorganisms are microalgae, in many cases, it is necessary to supply carbon dioxide thereto for culturing. In a case of dispersion culture using a conventional method, carbon dioxide is supplied through bubbling or the like using a pipe, but in this case, it is necessary to install long large pipe and to control the supply of carbon dioxide at high accuracy. Still another object of the present invention is to provide a method for efficiently supplying carbon dioxide.

Furthermore, in order to culture microorganisms, it is necessary to effectively use land and to improve the harvested amount of microorganisms per unit area. Still another object of the present invention is to improve such a task.

Furthermore, regarding photosynthetic microorganisms such as microalgae, in a case of using sunlight as a light source, a large quantity of microalgae is wasted since the intensity of solar radiation is too strong, and therefore, a decrease in proliferation rate, which is thought to be caused by photolesion can also be seen. Still another object of the present invention is to improve such a task.

Furthermore, in a case where microorganisms are made to proliferate between a substrate and water-absorbent polymer gel, depending on the type of microorganism or substrate, it can be seen that gaseous substance is released in accordance with the progress of proliferation, bubbles are formed in a region between the substrate and the water-absorbent polymer gel layer, drying of microorganisms existing in the bubbles is progressed, and therefore, the amount of collected substance of microorganisms, which is derived from decrease in proliferation rate or from strong adhesion of microorganisms on the substrate, is decreased. Still another object of the present invention is to provide a method for improving such a problem.

Furthermore, in a case where the culture process is performed plural times with respect to water-absorbent polymer gel, decrease in the amount of proliferation which is thought to be caused by decrease in nutrient components is observed. Still another object of the present invention is to improve such a problem.

Furthermore, in a case of newly starting culturing or repeatedly performing culturing, it is necessary to prepare seed algae. The preparation of the seed algae is extremely complicated and it is necessary to prepare a culture vessel for seed algae. This is also a cause of increased costs of culturing. Still another object of the present invention is to improve such a problem.

Furthermore, even in the case of making microorganism proliferate between a substrate and water-absorbent polymer gel, the moisture content of a collected substance of microorganisms is not less than or equal to 60%. Still another problem of the present invention is to provide a method for further obtaining a collected substance of microorganisms with low moisture content.

In addition, still another object of the present invention is to provide a culture method according to the present invention, a manufacturing method for obtaining a useful substance from microorganisms obtained through a collection method.

That is, the objects of the present invention is to provide methods for culturing and collecting microorganisms in which microorganisms are made to proliferate and are cultured between a substrate and the top of the surface of a gel formed of a water-absorbent polymer compound, the substrate is peeled off from the surface of the gel, and then, microorganisms are collected from the surface of the substrate; for applying the culture method to a wall surface culture method; for providing a structure, through which gas can easily pass, in a part of the substrate; and for decreasing the moisture content by drying a substrate, to which a collected substance of microorganisms is adhered, in the air, or by naturally drying the collected substance in the air.

The present inventors have conducted extensive studies in order to solve the above-described problems, and as a result, they have found that it is possible to peel off a proliferation substance from a gel layer in a state in which a large amount of the proliferation substance is made to be adhered to a substrate or a water-absorbent gel after culturing microorganisms in a region between the substrate and the gel layer formed of a water-absorbent polymer compound containing nutrient components enabling proliferation of microorganisms, and to collect the proliferation substance in a state of being low in moisture content. Furthermore, they have found that it is possible to obtain a useful substance from the microorganisms collected in this manner. The present invention has been completed based on these findings.

The present invention provides the followings.

[1] A method for culturing microorganisms,

in which microorganisms are cultured between at least a part of the surface of water-absorbent polymer gel, on which it is possible to culture microorganisms and which contains nutrients and water, and a substrate with which it is possible to coat the part of the surface.

[2] The culture method according to [1],

in which microorganisms Bawl a biofilm through culturing.

[3] The culture method according to [1] or [2], including:

a step of seeding microorganisms on at least a part of the surface of the water-absorbent polymer gel;

a step of coating a region on the water-absorbent polymer gel on which at least microorganisms are seeded, with the substrate; and

a step of culturing the seeded microorganisms between the substrate and the surface of the water-absorbent polymer gel.

[4] The culture method according to [1] or [2],

in which the microorganisms are microalgae which can be subjected to liquid surface-floating culture, and the seeding of microalgae on the surface of the water-absorbent polymer gel is performed by coating the at least the part of the surface of the water-absorbent polymer gel with a substrate to which a biofilm formed on the liquid surface through liquid surface-floating culture is transferred, or by transferring the biofilm formed on the liquid surface through liquid surface-floating culture to the surface of the water-absorbent polymer gel.

[5] The culture method according to [1] or [2],

in which seeding of microorganisms on the surface of the water-absorbent polymer gel is performed by coating the at least the part of the surface of the water-absorbent polymer gel with a substrate which has been immersed in a microorganism suspension liquid, or by immersing the surface of the water-absorbent polymer gel in the microorganism suspension liquid.

[6] The culture method according to [1] or [2],

in which seeding of microorganisms on the surface of the water-absorbent polymer gel is performed by spraying or coating at least either of the surface of the water-absorbent polymer gel or the substrate with microorganisms.

[7] The culture method according to any one of [1] to [6],

in which the culturing is performed using both surfaces of the water-absorbent polymer gel.

[8] The culture method according to any one of [1] to [7], further including:

a step of collecting microorganisms after the culturing.

[9] The culture method according to [8], further including:

a step of re-using the water-absorbent polymer gel or the substrate in culturing after the collection of microorganisms.

[10] The culture method according to [9],

in which the reuse of the water-absorbent polymer gel is performed after adding a fresh medium.

[11] The culture method according to any one of [8] to [10], further including:

a culture step in which microorganisms remaining on the water-absorbent polymer gel or the substrate after the collection of microorganisms, are used as seed microorganisms.

[12] The culture method according to any one of [8] to [11], further including:

a step of reducing the moisture content while maintaining an obtained collected substance to be adhered to the substrate or after making the collected substance be detached from the substrate,

in which the collection of the microorganisms after the culturing is performed by removing the substrate, to which the microorganisms are adhered, from the water-absorbent polymer gel.

[13] The culture method of microorganisms according to any one of [1] to [12],

in which the carbon dioxide permeability of the substrate is greater than or equal to 500 cc/m²·24 h/atm.

[14] The culture method according to [13],

in which the material of the substrate is at least one selected from the group consisting of polyethylene, polystyrene, polyester, nylon, polyvinyl chloride, and silicone rubber.

[15] The culture method according to any one of [1] to [14],

in which the culture method is vertical culture, in which the surface of the water-absorbent polymer gel is maintained in a vertical direction, or horizontal culture in which the surface of the water-absorbent polymer gel is maintained in a horizontal direction.

[16] The culture method according to any one of [1] to [15],

in which holes are bored at least at one or more sites on the substrate.

[17] The culture method according to any one of [1] to [16],

in which an uneven structure is formed in at least a part of a region on at least one of the substrate and the water-absorbent polymer gel.

[18] The culture method according to any one of [1] to [17],

in which at least a part of the surface of the water-absorbent polymer gel is coated with a plurality of substrates.

[19] The culture method according to any one of [1] to [18],

in which the microorganisms are eumycetes, green algae, or diatoms.

[20] The culture method according to any one of [1] to [19],

in which the microorganisms belong to yeast, Botryococcus sp., Chlamydomonas sp., Chlorococcum sp., Chlamydomonad sp., Tetracystis sp., Characium sp., Protosiphon sp., or Haematococcus sp.

[21] The culture method according to any one of [1] to [20],

in which the microorganisms belong to the same species as that of Botryococcus sudeticus or Chlorococcum sp. FERM BP-22262.

[22] The culture method according to any one of [1] to [21],

in which the microorganisms are Botryococcus sudeticus FERM BP-11420 or microalgae strains having taxonomically the same properties as those of Botryococcus sudeticus FERM BP-11420, or are Chlorococcum sp. FERM BP-22262 or microalgae strains having taxonomically the same properties as those of Chlorococcum sp. FERM BP-22262.

[23] A method for manufacturing biomass, including:

a culture step including the culture method according to any one of [1] to [22]; and

a step of collecting a biofilm on the liquid surface formed through a second culture step.

[24] The manufacturing method according to [23],

in which the biomass is oil.

[25] Microorganisms of which the identity with base sequences of a partial region corresponding to Chlorococcum sp. RK261 among base sequences encoding a gene region of 18S rRNA is 95.00% to 99.99% or which belong to Chlorococcum sp.,

in which the 18S rRNA gene thereof has sequence identity of at least 99.94% with polynucleotide formed of a base sequence of SEQ ID No: 2.

[26] Microorganisms which are Chlorococcum sp. FFG039 strains (accession number of FERM BP-22262) or microorganisms having taxonomically the same properties as those of Chlorococcum sp. FFG039 strains.

The culture method of the present invention is a method for culturing microorganisms in a region between a substrate and a gel formed of a water-absorbent polymer compound containing nutrient components enabling proliferation of microorganisms. Since the present invention employs such a form of culturing, it is possible to culture microorganisms with a minimum amount of water used, without performing stirring at all. Furthermore, since the method is culturing in such a narrow region, a biofilm which enables culturing at high density is formed. Accordingly, it is unnecessary to collect microorganisms from a large amount of medium, and thus, it is extremely easy to collect microorganisms. Furthermore, in the process of peeling off a substrate from a gel layer, in many cases, a large amount of the proliferation substance is adhered to the substrate side which has a higher intensity compared to the surface of the water-absorbent polymer gel layer. Therefore, it is possible to collect the proliferation substance from the substrate side which is easily handled, and thus, it is easy to perform the collection. Furthermore, in the method of the present invention, culturing can be performed while rarely depending on the type of microorganism. Furthermore, it is considered that a large amount of moisture exists in the gel layer and only minimum moisture required for proliferation is contained in the microorganism layer. Therefore, the moisture content in the collected substance of microorganisms becomes significantly low, and thus, it is possible to dramatically decrease energy to be input to a drying process. In addition, since the culture region is coated with the substrate, the present invention is also strong against an invasion of non-target microorganisms which causes a problem when performing outdoor culturing. Furthermore, by using a substrate which has high carbon dioxide permeability, it is possible to efficiently supply carbon dioxide to microorganisms from a gas phase. Moreover, unlike in the conventional method, it is unnecessary to provide long large pipe and to control the supply of carbon dioxide at high accuracy, and therefore, the culturing can be performed at low costs.

Since the water-absorbent polymer gel used in the present invention is a semisolid medium, it is possible to perform wall surface culturing and to significantly increase the quantity of microorganisms collected per culture device installation area. That is, it is possible to effectively use land. Furthermore, it is also possible to perform double-wall-surface culture by providing gel layers to both surfaces of a support substrate. Accordingly, it is possible to further achieve efficiency of installation area. In general, the support substrate also plays a role of holding a water-absorbent polymer gel layer having weak strength. In a case of using microalgae as microorganisms, it is possible to guide light to an air layer, that is, disperse light, and therefore, it is possible to effectively use the amount of light. Accordingly, it is possible to avoid photolesion under high intensity of light and to improve the proliferation rate. Furthermore, with the provision of a structure, through which gas can pass, in at least a part of a substrate, in a case where a gaseous substance is generated from microorganisms, the gaseous substance can be escaped outside a culture vessel. Therefore, it is possible to avoid various problems derived from the generation of gas.

It is possible to reuse a water-absorbent polymer gel layer by adding a medium, which favorably promotes proliferation of microorganisms, to the water-absorbent polymer gel layer which has been used once. Accordingly, it is unnecessary to newly prepare a water-absorbent polymer gel layer, and it is possible to efficiently perform culturing at low costs. It is also possible to reuse a substrate. In addition, it is possible to further improve the above-described effect by adding a medium at a higher concentration than the components of a medium which has been used in dispersion culture as a conventional method. Furthermore, it is possible to efficiently replace a medium in water-absorbent polymer gel in combination with a prescription of reducing the moisture content in the water-absorbent polymer gel.

After the collection of a microorganism layer, very small quantity of microorganisms remains on the surface of the gel or the surface of the substrate. In the present invention, by using these remaining microorganisms, it is possible to continue the culturing without supplying new seed algae. Furthermore, in a case where microorganisms after the culturing are collected while being adhered to the substrate, it is possible to perform drying treatment while leaving the microorganisms intact. In addition, even with the collected substance immediately after the collection from the substrate after the culturing, the moisture content is about 60%. Thus, the shape of the collected substance is in an irregular shape and the surface area is large. Therefore, even in this state, it is possible to efficiently dry the collected substance within a short period of time. That is, it is possible to easily obtain a collected substance with low moisture content using these methods.

### BRIEF DESCRIPTION OF THE DRAWINGS

FIGS. 1A to 1H are schematic views of the present invention. Reference Numeral 1 represents water-absorbent polymer gel, Reference Numeral 2 represents a support substrate, Reference Numeral 3 represents a microorganism layer, Reference Numeral 4 represents a substrate, Reference Numeral 5 represents microorganisms proliferating through culturing, and Reference Numeral 6 represents collected microorganisms. FIG. 1A shows a state in which a water-absorbent polymer gel layer 1 including a medium capable of favorably performing culturing of microorganisms, to be cultured, on the support substrate 2. FIG. 1B shows a state in which the water-absorbent polymer gel layer is coated with the microorganism layer 3. FIG. 1C shows a state in which the microorganism layer 3 is coated with the substrate 4. FIG. 1D is a view showing that the quantity of microorganisms is increased through proliferation of the microorganisms as a result of culturing the microorganisms under the conditions at which the microorganisms can favorably proliferate. FIG. 1E is a view illustrating a state in which almost all of the microorganisms 5 are adhered to the substrate 4 side in a state in which the substrate 4 is peeled off from the water-absorbent polymer gel layer 1. FIG. 1F is in the same state as that of FIG. 1A, but in general, a small quantity of microorganisms is adhered thereto. FIG. 1G shows a state of microorganisms after the microorganisms are detached from FIG. 1E. FIG. 1H shows a substrate in a state in which the microorganisms are detached from FIG. 1E, but in general, a few microorganisms are adhered thereto.

FIGS. 2A and 2B are schematic views in a case where culturing is started after microorganisms are adhered to a substrate. Reference Numeral 1 represents water-absorbent polymer gel, Reference Numeral 2 represents a support substrate, and Reference Numeral 4 represents a substrate. FIG. 1A shows a state in which microorganisms are adhered to the substrate 4 and FIG. 1B shows a state in which the water-absorbent polymer gel is coated with the substrate to which the microorganisms are adhered.

FIGS. 3A to 3F are schematic views of a liquid surface-floating culture method. Reference Numeral 4 represents a substrate, Reference Numeral 7 represents a culture vessel, and Reference Numeral 8 represents a microorganism suspension liquid. FIG. 3A shows a state in which the microorganism suspension liquid is put into the culture vessel, FIG. 3B shows a state in which microorganisms are sunk to the bottom surface of the culture vessel after allowing the state of FIG. 3A to stand for several seconds to several tens of minutes, FIG. 3C shows a state in which a biofilm of microorganisms is formed on the liquid surface when the culturing is continued for a while, FIG. 3D shows a state in which the biofilm of microorganisms on the liquid surface is coated with a substrate, FIG. 3E shows a state in which the microorganism-adhered substrate is moved outside the culture vessel, and FIG. 3F shows a state of the culture vessel after the microorganism-adhered substrate is removed from the culture vessel.

FIGS. 4A to 4L are schematic views of a double-surface culture method. Reference Numeral 1 represents a water-absorbent polymer gel layer, Reference Numeral 3 represents microorganisms before culturing, Reference Numeral 4 represents a substrate (support substrate), Reference Numeral 5 represents microorganisms after proliferation, and Reference Numeral 6 represents microorganisms detached from the substrate. FIG. 4A shows a state in which the water-absorbent polymer gel layer is adhered to the substrate, FIG. 4B shows a state in which microorganisms are adhered to the water-absorbent polymer gel, FIG. 4C shows a state in which the microorganism layer is coated with a substrate, FIG. 4D shows a state in which the substrate having no microorganism layer is removed from the water-absorbent polymer gel layer, FIG. 4E shows a state in which microorganisms are adhered to the water-absorbent polymer gel, FIG. 4F shows a state in which the microorganism layer is coated with a substrate, FIG. 4G shows a state after culturing is performed, FIGS. 4H and 41 show a state in which microorganisms on one side are peeled off together with the substrate, FIG. 4J shows a state in which microorganisms are adhered to the water-absorbent polymer gel and are coated with a substrate, FIG. 4K shows a state in which the substrate on the other side is peeled off together with microorganisms which have proliferated, and FIG. 4L shows a state, in which microorganisms are adhered to the water-absorbent polymer gel and are coated with a substrate, and is in substantially the same state as that of FIG. 4F.

FIG. 5 is a schematic view in a case where water-absorbent polymer gel layers are installed on both sides of a support substrate. This method is used in the case of double-surface culture. Reference Numeral 1 represents a water-absorbent polymer gel layer and Reference Numeral 2 represents a support substrate.

FIG. 6 is a composition of a CSiFF03 medium.

FIG. 7 is a composition of a CSiFF04 medium.

FIG. 8 shows the quantity of dry alga bodies (bar graph) and the moisture content (white blank circle) in a case where microalgae are cultured using water-absorbent polymer gel (agarose gel) and a medium (liquid medium) for culturing microorganisms.

FIG. 9 shows the quantity of dry alga bodies in a case where culturing is restarted after adding a fresh medium to water-absorbent polymer gel (agar medium) which has been used for culturing once.

FIG. 10 shows the quantity of dry alga bodies in a case where culturing is performed under various experimental conditions. 4-1 is a case where agarose gel is coated with algal bodies (case where the algal bodies are not coated with a substrate). 4-2 is a case where agarose gel is coated with algal bodies, which are then coated with a substrate. 4-3 is a case where the surface of agarose gel is coated with a substrate to which microalgae are adhered. 4-4 is a case where a microalga-adhered substrate is pasted on a support substrate (case where there is no water-absorbent polymer gel).

FIG. 11 is the moisture content in cases where culturing is performed under various experimental conditions. The description is the same as that in FIG. 10.

FIG. 12 is an influence of the type of material of a substrate on the quantity of dry alga bodies.

FIG. 13 is an influence of the type of material of a substrate on the moisture content.

FIGS. 14A to 14C show a state at the time of collection after culturing in the present invention. FIG. 14A shows a state in the end of the culturing and FIG. 14B shows a state in which a substrate (silicone rubber sheet) is peeled off after the completion of the culturing. In FIG. 14B, the left side is agarose gel after the substrate is peeled off, and the right side is a silicone rubber sheet after the substrate is peeled off. It can be seen that microalgae are adhered to the substrate. In FIG. 14B, a film to which the microalgae are attached is placed on a lid of a plastic Petri dish. FIG. 14C shows a state after microalgae adhered to the substrate are peeled off from the substrate (two samples).

FIGS. 15A to 15D show a state of vertical culture. FIG. 15A shows an entire state of vertical culture and shows a state 7 days after the start of the culturing. Both sides are agarose gel layers to which no microalgae are adhered, and two at the center are agarose gel layers to which microalgae are adhered. FIG. 15B shows an agarose gel-AVFF007 strain-silicone rubber sheet structure 7 days after the start of the culturing. FIG. 15C shows an AVFF007 strain-silicone rubber sheet structure after being peeled off from the agarose gel 7 days after the start of the culturing. FIG. 15D shows the agarose gel after the substrate is peeled off therefrom.

FIG. 16 shows a difference between horizontal culture and vertical culture per culture area.

FIG. 17 shows a difference between horizontal culture and vertical culture per installation area.

FIG. 18 shows an influence of the difference between double-surface culture and single-surface culture on the amount of proliferation of microalgae.

FIG. 19 is a schematic view of a film bored with a hole.

FIG. 20 shows influences of a case where a substrate is bored with a hole and a case where the substrate is not bored with a hole, on the amount of proliferation of microalgae.

FIG. 21A shows a state immediately after a film is separated from agarose gel in the case where the film has not been bored with a hole; FIG. 21B\ shows a state on the agarose gel after the operation of FIG. 21A has been performed; FIG. 21C shows a state of the agarose gel after a film bored with a hole is separated from the agarose gel after the culturing using this film.

FIG. 22 is a part of a base sequence (SEQ ID No: 1) of a gene which encodes 18S rRNA of Botryococcus sudeticus AVFF007 strains of microalgae.

FIGS. 23A and 23B show microscopic photographs of Chlorococcum sp. FFG039 strains. FIG. 23A shows a general state and FIG. 23B shows a state in which zoospores are released and proliferated.

FIG. 24 is a gene sequence of the FFG039 strains obtained through 18S rDNA analysis.

FIG. 25 is a genealogical tree of the FFG039 strains.

### DESCRIPTION OF THE PREFERRED EMBODIMENTS

Hereinafter, a preferred embodiment of a method for culturing microorganisms according to the present invention will be described in detail. The numerical range represented by “˜” means a range including numerical values denoted before and after “˜” as a lower limit value and an upper limit value.

### METHOD OF PRESENT INVENTION

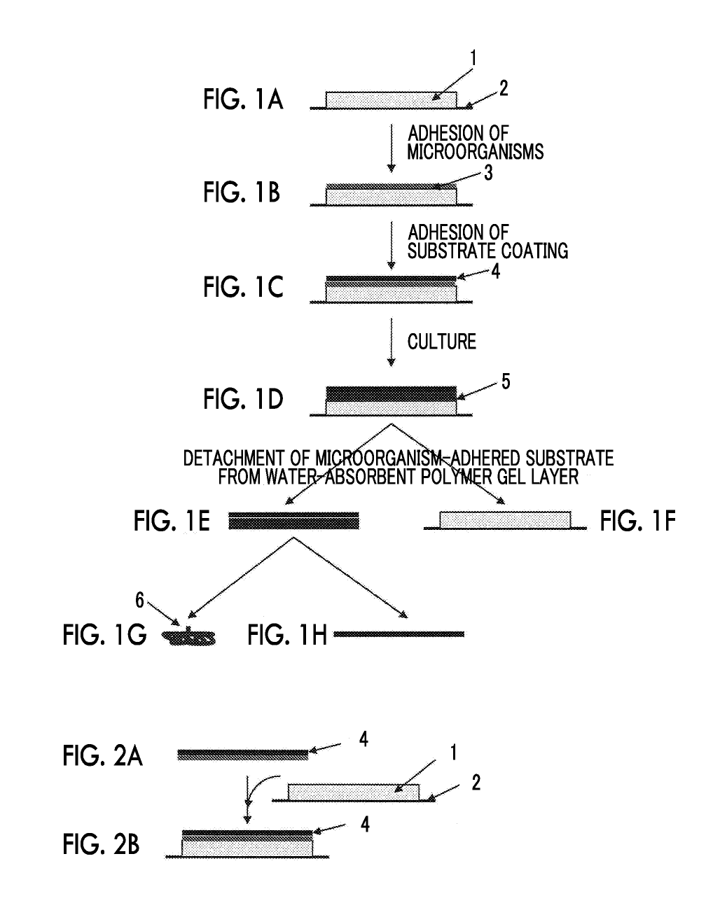

A basic culture method of the present invention is shown in FIGS. 1A to 1H. This schematic view is provided in order to describe the present invention, and therefore, some sections of this drawing are denoted by being simplified.

As shown in FIG. 1A, water-absorbent polymer gel 1 is formed on a support substrate 2. The water-absorbent polymer gel 1 may be moved to the top of the support substrate after being formed. In addition, after forming a projection body on a substrate, the entirety of or at least a part of the projection body may be coated with water-absorbent polymer gel. Accordingly, in some cases, the form of the water-absorbent polymer gel which generally has a weak strength is stabilized and the adhesiveness between the water-absorbent polymer gel and the support substrate is improved.

The water-absorbent polymer gel 1 may be impregnated with a medium after forming a gel layer. However, it is preferable that the water-absorbent polymer gel is impregnated with the medium simultaneously with the formation of the gel layer since the time for impregnation becomes short and it is possible to uniformly distribute a nutrient source.

However, in a case of re-using the water-absorbent polymer gel 1 (process from FIG. 1F to FIG. 1B), it is preferable to add a medium to the water-absorbent polymer gel. A medium having the same composition as that in pre-culture may be used, or a medium formed of a composition of a different medium may be used. In the latter case, a medium of which the proportion of the medium components and each component is the same as that in the pre-culture, but the concentration is different from that in the pre-culture can also be used. For example, there is a medium which is prepared by making the concentration of all compositions forming the medium be twice the concentration in the pre-culture. By doing this, it is possible to impregnate the water-absorbent polymer gel 1 with a larger amount of nutrient components. In addition, the water-absorbent polymer gel may be impregnated with a medium after performing drying treatment on the water-absorbent polymer gel 1. By doing this, it is possible to more quickly impregnate the water-absorbent polymer gel 1 with a medium.

As shown in FIG. 1B, the surface of the water-absorbent polymer gel is coated with microorganisms. As the coating method, any well-known method may be used. For example, there is a method for adding dropwise a medium containing microorganisms to the surface of the water-absorbent polymer gel using a pipette, a method for thinly spreading the medium which has been added dropwise on the surface of the water-absorbent polymer gel layer, and a method for coating the surface thereof through spin coating. In addition, it is also possible to spray, a solution containing microorganisms in a mist form. By doing this, a seed microorganism layer 3 is formed and enters a state of in FIG. 1B. The solution containing microorganisms may be subjected to or not be subjected to suspension treatment. However, the solution thereof is more preferably subjected to the suspension treatment since it is possible to uniformly distribute microorganisms on the surface of the water-absorbent polymer gel layer. The surface of the water-absorbent polymer gel layer 1 is preferably flat, but may have unevenness. This is because, with the provision of unevenness, carbon dioxide can diffuse through a gap generated between the substrate and the surface of the gel. In addition, in the drawing, it is shown such that the surface of the gel is uniformly formed. The surface may have unevenness, but is preferably uniform as much as possible.

Next, as shown in FIG. 1C, the surface of the seed microorganism layer 3 is coated with a substrate 4. A microorganism layer 5 is formed as shown in FIG. 1D by performing culturing in this state. In a case where it is determined that this layer sufficiently proliferates, the substrate is peeled off from the water-absorbent polymer gel layer 1. This state is shown by states of FIGS. 1E and 1F. In this schematic view, microorganisms are adhered to the FIG. 1E side, but may be adhered to the FIG. 1F side or both the sides. It is desirable that microorganisms are adhered to one side from the viewpoint of collection efficiency, and in particular, it is more preferable that microorganisms are adhered to the FIG. 1E side. This is because it is preferable to collect microorganisms from the substrate 4 with strong strength compared to collecting microorganisms from the water-absorbent polymer gel layer 1 which is generally thought to have weak surface strength in a case where microorganisms are collected using a device such as a solid holding device, from the viewpoint of re-using the water-absorbent polymer gel layer 1. The adhesion of microorganisms to the substrate 4 or the water-absorbent polymer gel layer 1 indicates a state in which most microorganisms are adhered to either side, but does not indicate a state in which there is no microorganism at all on a non-adhesive surface.

In a case of collecting microorganisms from the surface of the substrate, it is possible to collect the microorganisms on the surface in FIG. 1E using a cell scraper. Accordingly, the substrate in FIG. 1H and the collected substance 6 in FIG. 1G are obtained. In some cases, microorganisms are adhered to the water-absorbent polymer gel layer in Fig. F, but the schematic view of the case is omitted.

The substrate 4 or the water-absorbent polymer gel layer 1 does not enter a state in which microorganisms do not exist at all, even after microorganisms are detached from the substrate or the water-absorbent polymer gel layer. Accordingly, by using the microorganisms existing on the surfaces thereof as seed algae, it is possible to start culturing by sticking the water-absorbent polymer gel layer 1 and the substrate 4 on each other without coating the surfaces with microorganisms.

In addition, the microorganism layer 5 may be detached from the substrate after decreasing the moisture content by performing a drying process in the state of FIG. 1E. Furthermore, a collected substance may be obtained after reducing the moisture content by performing drying process in the state of FIG. 1G Furthermore, a collected substance after being dried may be obtained using both methods. That is, algal bodies on the substrate may be dried, the dry alga bodies may be detached from the substrate, and the detached dry alga bodies may be further dried. As the drying process, it is possible to use any well-known method such as drying through heating, freeze-drying, and natural drying using sunlight. Natural drying using sunlight is most preferable. In the state of FIG. 1G, in many cases, the moisture content is less than or equal to 70%. In this case, the algal bodies are in an irregular shape and the surface area becomes extremely large. For this reason, it is possible to efficiently perform the drying. In a case where it is expected that the moisture content of the collected substance 6 in FIG. 1G is high, in some cases, it is difficult to obtain such an effect. In this case, a drying process may be performed in the state of FIG. 1E.

In the present invention, it is possible to start culturing after making microorganisms adhere to the substrate 4. The schematic view in the case of performing such a culture method is shown in FIGS. 2A and 2B. As shown in FIG. 2A, microorganisms are adhered to the surface of the substrate 4. As the method, any well-known method may be used. Examples thereof include a method for coating the surface of the substrate 4 with a microorganism suspension liquid; a method for immersing the substrate in a suspension liquid of microorganisms and making the microorganisms adhere to and deposit on the substrate; and a method for forming a biofilm of microorganisms on the liquid surface by performing liquid surface-floating culture and making the biofilm adhere on the surface of the substrate. Next, as shown in FIG. 2B, culturing is performed after coating the surface of the water-absorbent polymer gel 1 with a microorganism-adhered substrate. This drawing and the FIG. 1C are the same as each other, and the remaining process is the same as that performed after the FIG. 1C. The water-absorbent polymer gel layer 1 and the substrate 4 may be stuck on each other after making microorganisms adhere to both the water-absorbent polymer gel layer and the substrate.

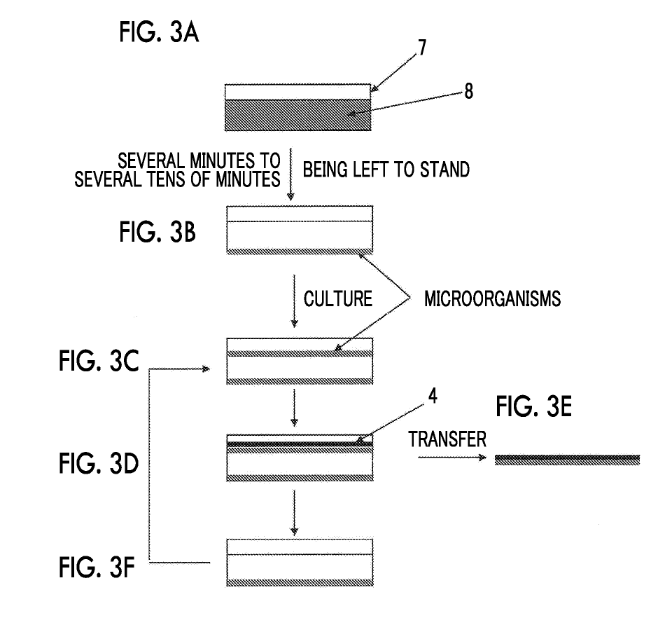

In FIG. 3A to FIG. 3F, a method for forming a biofilm of microorganisms on the liquid surface through liquid surface-floating culture and transferring the biofilm on a substrate to prepare the substrate to which microorganisms are adhered. As shown in FIG. 3A the suspension liquid 8 of microorganisms is prepared and put into a culture vessel. Next, when the culture vessel is in a stationary state, as shown in FIG. 3B, the microorganisms sink to the bottom surface of the culture vessel 7 within several seconds to several tens of minutes depending on the types of microorganisms. The sinking of microorganisms to the bottom surface means that the majority of microorganisms sink to the bottom surface, and does not mean a state in which microorganisms completely disappear from the top of the liquid surface, from the middle of the solution, the side surface of the culture vessel, or other surfaces, in a medium. When the stationary culture is performed in this state for a while, a biofilm formed of the microorganisms is formed on the liquid surface as shown in FIG. 3C. The structure of the biofilm changes from a film-like structure to a three-dimensional structure when the culturing is continuously performed. The change is continuous. In addition, as shown in FIG. 3C, the microorganisms also exist on the bottom surface of the culture vessel. Moreover, although it is not shown in the drawing, the microorganisms also exist on the side surface of the culture vessel or other surfaces.

Next, as shown in FIG. 3D, the biofilm of microorganisms are adhered to the top of the substrate through a transferring method. The state in which the microorganism-adhered substrate is taken out of the culture vessel is a state of FIG. 3E. This state is the same as that of FIG. 2A and the culturing performed in the process shown in FIGS. 1A to 1H can be performed after the FIG. 3E. The state after the microorganisms on the liquid surface are removed through FIG. 3D is FIG. 3F. There are microorganisms on the bottom surface or the side surface within the culture vessel and even on the liquid surface, and therefore, it is possible to newly start culturing therefrom. As a result, it is possible to return to the state in FIG. 3C. This cycle can be performed without any limitation as long as there are nutrient components for proliferation remaining in the medium. Furthermore, after the entirety or a part of the medium is removed, it is possible to perform culturing many times by adding a fresh medium.

In the present invention, as shown in FIG. 3A to FIG. 3F, it is possible to prepare the microorganism-adhered substrate shown in of FIG. 2A by transferring the biofilm formed on the liquid surface using a substrate. As the biofilm on the liquid surface, it is possible to use a three-dimensional structure which is obtained from a part of a film-like structure rising in a bubble shape in accordance with the progress of the culturing. It is preferable to use the film-like structure from the viewpoint of securing room for improving proliferation in a primary culturing process. However, there is no limitation.

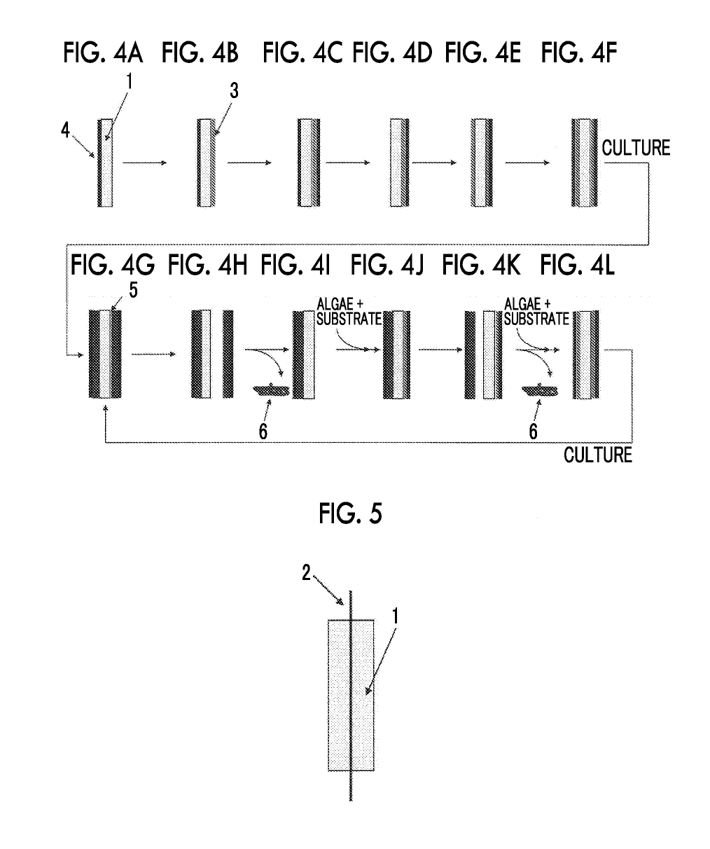

In FIGS. 4A to 4L, a schematic view in a case where wall surface culture is performed is shown. In this schematic view, a case where double-surface culture is performed is shown. However, it is possible to perform culturing by performing several changes, even in a case of a single-surface culture. FIG. 4A shows a structure of water-absorbent polymer gel 1 and a substrate 4. Only the water-absorbent polymer gel 1 may be used, but use of the substrate 4 together is preferably from the viewpoint of strength since the water-absorbent polymer gel generally has a weak strength. In addition, the substrate 4 also plays the same role as that of the support substrate 2. FIG. 4B shows a structure in which the surface of the water-absorbent polymer gel 1 on a side opposite to the substrate is coated with microorganisms. Similarly to FIGS. 1A to 1H, any well-known method may be used as the coating method. FIG. 4C shows a structure in which the surface coated with the microorganisms is coated with the substrate 4. As shown in FIGS. 2A and 2B, the water-absorbent polymer gel 1 may be coated with the substrate 4 to which the microorganisms are adhered. In addition, both the substrate 4 and the water-absorbent polymer gel 1 may be stuck on each other after being coated with microorganisms. Next, as shown in FIG. 4D, the substrate on the side opposite to the surface coated with microorganisms is peeled off from the water-absorbent polymer gel. The substrate 4 coated with microorganisms in FIG. 4D also plays a role as a support of the water-absorbent polymer gel.

Next, after coating the water-absorbent polymer gel with microorganisms as shown in FIG. 4E and coating the water-absorbent polymer gel 1, which is coated with microorganisms, with the substrate 4 as shown in FIG. 4F, the culturing is continuously performed. The result of performing the culturing is FIG. 4G After the culturing, a one substrate is peeled off as shown in FIG. 4H and microorganisms are detached from the substrate using a cell scraper or the like. Reference. Numeral 6 is a collected substance thereof. In FIG. 1H, the substrate on the right side of the water-absorbent polymer gel 1 is first peeled off, but the substrate on the right side thereof may also be peeled off. In addition, the substrates may be peeled off at the same time. However, it is preferable to peel off one by one since the water-absorbent polymer gel generally has a low strength. In addition, in the drawing, it is shown such that microorganisms are adhered to the substrate 4 side. However, the same procedure can be performed, even if microorganisms are adhered to the water-absorbent polymer gel 1 side. Microorganisms and a substrate 4 are pasted on the surface of the water-absorbent polymer gel 1 on the side on which the substrate 4 is removed as shown in FIG. 4I. Microorganisms, with which the water-absorbent polymer gel 1 is coated, may be coated with the substrate 4, or the water-absorbent polymer gel may be coated with the substrate which is coated with microorganisms. Through this process, the structure enters a state of FIG. 4J. The microorganisms on the other side are collected as shown in FIG. 4K and microorganisms are adhered to the water-absorbent polymer gel again (FIG. 4L), and the structure enters a state of FIG. 4G by performing culturing. This process may be repeated many times. The number of microorganisms 3 is smaller than the number of microorganisms 5. In addition, only one structure formed of the substrate, the water-absorbent polymer gel, and microorganisms is shown in the drawing; but a plurality of structures may be arranged and used. In addition, in the drawing, the structure is shown so as to be in a vertical direction with respect to the ground, but may be installed at any angle. In a case of installing a plurality of structures, the installation angle or the size of each structure, the thickness of each water-absorbent polymer gel 1, the type of each substrate 4, or the like may vary.

In addition, in the present invention, it is also possible to perform culturing by pasting the microorganism-adhered substrate prepared in FIG. 3E to the water-absorbent polymer gel 1.

In FIGS. 4A to 4L, the substrate 4 adhered to the surface of the water-absorbent polymer gel 1 is used as a support of the water-absorbent polymer gel 1. However, the substrate 2 as a support may be installed inside a water-absorbent polymer gel as shown in FIG. 5. In this substrate, the portion which is coated with the water-absorbent polymer gel is basically not brought into contact with microorganisms and functions only as the support. The support substrate 2 preferably has a higher strength than that of the substrate 4. This is because, the former requires strength for functioning as a support of the structure, but the latter also functions as a substrate for collection, and therefore, in some cases, it is necessary to have flexibility. In addition, the support substrate 2 may have a penetrating structure. Accordingly, the water-absorbent polymer gel 1 becomes a continuous structure through the penetrating structure, and therefore, it is easy to maintain the form of the structure.

[Microorganisms which can be Cultured in Present Invention]

As microorganisms which can be used in the present invention, any type of microorganism is used as an object, as long as various well-known culture methods, for example, floating-medium surface floating-culture or adhesive culture, can be performed, and as long as culturing using a medium which can be artificially prepared can be performed.

The microorganisms of the present invention indicate minute organisms of which the individual existence cannot be identified with the human naked eye. As the microorganisms, it is possible to use algae as eukaryotes, protoctists, fungi, myxomycetes as well as eubacteria and archaebacteria. In addition, when microorganisms are referred in the present invention, plant cells and animal cells are included therein.

It is also possible to use microalgae as microorganisms. The above-described microalgae are not particularly limited. Either prokaryote or eukaryote may be used, and it is possible to appropriately select microalgae in accordance with the purpose. More specific examples thereof include the division Cyanophyta, the division Glaucophyta, the division Rhodophyta, the division Chlorophyta, the division Cryptophyta, the division Haptophyta, the division Heterokontophyta, the division Dinophyta, the division Euglenophyta, and the division Chlorarachniophyta. Among these, as the above-described microalgae, diatoms of the division Heterokontophyta and the division Chlorophyta are preferable, and the genus Haematococcus, the genus Chlamydomonas, the genus Chlorococcum, the genus Botryococcus, and the genus Nitzschia are more preferable in terms of production of biomass. These may be used alone or in a combination of two or more thereof.

The above-described method of obtaining microorganisms is not particularly limited, and any method can be appropriately selected in accordance with the purpose. Examples thereof include a method of collecting microorganisms in nature, a method of using a commercially available product, and a method of obtaining microorganisms from a culture collection or a depositary institution. Microalgae used in the present invention are preferably microalgae obtained through a purification process.

Yeast can also be used as microorganisms. The above-described yeast is not particularly limited, and examples thereof include yeast which belong to the genus Endomyces, the genus Eremascus, the genus Schizosaccharomyces, the genus Nadsonia, the genus Saccharomycodes, the genus Hanseniaspora, the genus Wickerhamia, the genus Saccharomyces, the genus Kluyveromyces, the genus Lodderomyces, the genus Wingea, the genus Endomycopsis, the genus Pichia, the genus Hansenula, the genus Pachysolen, the genus Citeromyces, the genus Debaryomyces, the genus Schwanniomyces, the genus Dekkera, the genus Saccharomycopsis, the genus Lipomyces, the genus Spermophthora, the genus Eremothecium, the genus Crebrothecium, the genus Ashbya, the genus Nematospora, the genus Metschnikowia, the genus Coccidiascus, or the genus Candida. These may be used along or in a combination of two or more thereof. Preferred examples of yeast which belong to the genus Candida include Candida utilis.

In the present invention, among the above-described microorganisms, microorganisms which can produce useful substances are preferable. Particularly, microorganisms which produce an intermediate or a final product for a pharmaceutical product, a cosmetic, or a health food product; a raw material used in synthetic chemistry; a hydrocarbon compound or triglyceride; an oily substance such as fatty acid compound; gas such as hydrogen; and the like are preferable. In the present invention, in some cases, these are called products. Furthermore, in the present invention, it is preferable to use microorganisms which satisfy either of good culturing on the liquid surface and good recovery from the liquid surface; possession of a high proliferation rate; possession of a high oil content ratio; generation of little odor at least during culturing; or no generation of poisonous substances being confirmed.

[Biofilm]

The biofilm in the present invention refers to a film-like structure or a steric three-dimensional structure to be described below, which is formed of microorganisms. In general, the biofilm thereof refers to a microorganism structure (microalgae aggregation or microalgae film) which is adhered to the surface of rock or the like. Besides these, in the present invention, the film-like structure or the three-dimensional structure, which is formed of microorganisms existing on the surface such as the liquid surface having fluidity, is also called a biofilm. A biofilm in nature also contains debris or pieces of plants besides target microorganisms. The biofilm of the present invention may contain these as long as the biofilm is a sample which has been obtained through a purification process. However, ideally, it is more preferable that the biofilm is foamed of only the microorganisms according to the present invention and a substance such as an intracellular matrix which is secreted during the proliferation of the microorganisms. In addition, microorganisms on the bottom surface of a culture vessel can also be called a biofilm as long as the microorganisms form a film-like structure.

In addition, it is preferable that the biofilm is configured such that individual microorganisms are adhered to each other directly or via a substance (for example, polysaccharides) such as the intercellular matrix. In general, in many cases, such a film-like structure is denoted as a biofilm or the like.

In addition, the purification process is a process which is performed for the purpose of making microalgae be a single type, and it is unnecessary to make microalgae be completely a single type.

In the present invention, the structure which proliferates between a water-absorbent polymer gel and a substrate and in which aggregations of microorganisms are substantially continued is also called a biofilm. In the present invention, at the time of completing a primary culturing process, it is desirable that a biofilm is formed in this region in view of obtaining a large amount of collected substance of microorganisms. In addition, when starting the primary culturing process, a biofilm structure may be or may not be formed in the region. In the case of the former, in many cases, it is possible to effectively use the region and to obtain a large amount of collected substance of microorganisms, which is more preferable.

[AVFF007 Strains]

The AVFF007 strains as microalgae used in Examples of the present specification are internationally deposited to the Patent Organism Depository of the National Institute of Advanced Industrial Science and Technology (Central 6, 1-1, Higashi 1-chome, Tsukuba-shi, Ibaraki-ken, Japan) dated Sep. 28, 2011 with a accession number of FERM BP-11420 under the Budapest Treaty, by FUJIFILM Corporation (26-30, Nishiazabu 2-chome, Minato-ku, Tokyo, Japan). The work of the Patent Organism Depository of the National Institute of Advanced Industrial Science and Technology was handed over to the Patent Organism Depositary of the National Institute of Technology and Evaluation (Room No. 120, 2-5-8 Kazusakamatari, Kisarazu-shi, Chiba-ken, Japan) from Apr. 1, 2012.

The AVFF007 strains are novel strains of freshwater microalgae which have been isolated from a freshwater pond in Kyoto, Japan by the present inventors. A part (SEQ ID No: 1, FIG. 22) of a base sequence of the 18S rRNA gene was analyzed using BLAST based on data of the National Center for Biotechnology Information (NCBI). As a result, the part of the base sequence thereof was identified as microalgae which were closely related to Botryococcus sp. UTEX2629 (Botryococcus sudeticus) strains (homologous to a 1109 base on the AVFF007 strain side among a 1118 base on the UTEX2629 strain side). The AVFF007 strains are microalgae which are also closely related to Characiopodium sp. Mary 9/21 T-3w, and there is also a possibility that the name of the AVFF007 strains may be changed to the genus Characiopodium. In this case, the name of the AVFF007 strains is regarded to be changed thereto. In addition, in a case where the name of the AVFF007 strains is changed to a name other than the genus Characiopodium, the name of the AVFF007 strains in the present invention is also regarded as being changed thereto.

In the present invention, it is possible to use strains having taxonomically the same properties as those of the AVFF007 strains. The taxonomic properties of the AVFF007 strains are shown below.

Taxonomic. Properties of AVFF007 Strains

1. Morphological Properties

The AVFF007 strains have a green circular shape. The AVFF007 strains have floating properties, and therefore, can proliferate on the liquid surface and the bottom surface. The size of an AVFF007 strain is 4 μm to 30 μm (the size of AVFF007 strain in a case of being present on the liquid surface is comparatively large, but the size of AVFF007 strain on the bottom surface is comparatively small). The AVFF007 strains proliferate on the liquid surface and form a film-like structure. Bubbles are formed on the liquid surface in accordance with the proliferation and overlap each other to form a three-dimensional structure on the liquid surface. In addition, oil is produced.

2. Culturing Properties (Culture Method)

(1) Medium: CSiFF04 (which is obtained by improving a CSi medium and of which the composition is shown in FIG. 7).

(2) Culture temperature: a favorable temperature is 23° C. and culturing can be performed at less than or equal to 37° C.

(3) The culture period (the period until the culturing generally reaches a stationary phase) depends on the quantity of algal bodies which has been initially used, and is 2 weeks to 1 month. In general, culturing can be performed at 10×10⁴ cells/mL.

(4) Culture method: aerobic culture and stationary culture are suitable.

(5) Light-requiring properties: Necessary. Intensity of light: 4000 lux to 15000 lux. Bright and dark cycle: 12 hours for time for a bright period and 12 hours for time for a dark period. During subculture, it is possible to perform the culturing at 4000 lux.

The AVFF007 strains can be stored through the subculture in accordance with the above-described culturing properties (culture method). The subculture can be performed by collecting microalgae which float on the liquid surface and performing dispersion such as pipetting, and then, dispersing the microalgae in a fresh medium. Immediately after the subculture, the microalgae are sunk on the bottom surface of a culture vessel, and start to form a biofilm on the liquid surface after about one week. Proliferation can be performed even if the microalgae exist on the liquid surface immediately after the subculture. The cycle for the subculture is about one month. Subculture is performed when the microalgae exhibit yellow color.

As strains having taxonomically the same properties as those of the AVFF007 strains, microalgae are included of which the 18S rRNA gene has sequence identity of at least 95.0%, preferably 98.0%, more preferably 99.0%, still more preferably 99.5%, and still more preferably 99.9% with polynucleotide formed of a base sequence of SEQ ID No: 1.

The sequence identity mentioned regarding the base sequence in the present invention means a percentage of the number of common bases which are coincident with each other between two arrayed bases within a region in a case where the two bases are arrayed in an optimal mode. That is, the identity can be calculated through an equation of identity=(number of coincident bases/total number of bases)×100 and can be calculated using an algorithm which is commercially available or open to the public. Search and analysis with respect to the identity of the base sequence can be performed using a well-known algorithm or program for those skilled in the art. In a case of using a program, those skilled in the art can appropriately set the parameter. Alternately, a default parameter of each program may be used. Specific techniques for the analysis method thereof are also well known to those skilled in the art.

[FFG039 Strains]

The FFG039 strains as microalgae used in Examples of the present specification are collected by the present inventors from Nara Prefecture in Japan. The FFG039 strains have proliferating properties and are excellent in oil productivity compared to the AVFF007 strains. In addition, the FFG039 strains have characteristics in that the structure of the biofilm is hardly destroyed and it is easy to collect the FFG039 strains. The FFG039 strains are Chlorococcum sp. As a result of analysis of the gene sequence of 18S rRNA, the FFG039 strains are species closely related to RK261 strains (Chlorococcum sp. RK261) of the genus Chlorococcum as microalgae. In the present invention, newly isolated microalgae are named Chlorococcum sp. FFG039. It is more preferable that the identity with base sequences of a partial region corresponding to RK261 of the genus Chlorococcum among base sequences encoding a gene region of the microalgae according to the present invention is 95.00% to 99.99%. The “partial region” referred to herein means a region having greater than or equal to 1000 base sequences. When testing the identity, use of every base sequence results in the highest reliability for the test of the identity. However, determining every base sequence is technically and financially difficult except for an extremely small number of species of organisms. In addition, only a specific portion (specifically, the vicinity of base sequences corresponding to base sequences of the Chlorococcum sp. FFG039 strains (hereinafter, also simply referred to as FFG039 strains) which are set as a comparison target to be described below) of the base sequences of the RK261 strains, of the genus Chlorococcum has not been disclosed. Furthermore, in general, it is considered that there can be attribution if about 1000 base sequences are read. From the above, the identity has been tested through the comparison with the base sequences of a “partial region” in the present invention, and it is considered that the reliability thereof is sufficiently high. The Japanese name of Chlorococcum is based on the Japanese name disclosed in Freshwater algae, written by Takaaki YAMAGISHI, UCHIDA ROKAKUHO PUBLISHING CO., LTD.

The FFG039 strains as microalgae used in Examples of the present specification are internationally deposited to the Patent Organism Depositary of the National Institute of Technology and Evaluation (Room No. 120, 2-5-8 Kazusakamatari, Kisarazu-shi, Chiba-ken, Japan) dated Feb. 6, 2014 with a accession number of FERM BP-22262 under the Budapest Treaty, by FUJIFILM Corporation (26-30, Nishiazabu 2-chome, Minato-ku, Tokyo, Japan).

The FFG039 strains are novel strains of freshwater microalgae which have been isolated from a pond in Kyoto in Japan by the present inventors and belong to the genus Chlorococcum.

Hereinafter, the method of isolating the microalgae (hereinafter, also referred to as purification) and circumstances in which it has been determined that the FFG039 strains of the microalgae are novel strains will be described.

[Purification of FFG039 Strains of Microalgae]

Natural fresh water was collected from a pond in Nara Prefecture by putting it into a 5 mL tube for homogenizing (TM-655S, Tomy Seiko Co., Ltd.). 100 μL of the collected natural fresh water was added to a 24 hole plate (microorganism culture plate 1-8355-02, As One Corporation) into which 1.9 mL of a medium, in which a CSiFF04 medium shown in FIG. 7 was put. The plate was placed on a plant bioshelf for tissue culture (AV152261-12-2, Ikeda Scientific Co., Ltd.) and was cultured at 23° C. under continuous irradiation with 4000 lux light. After approximately one month, a green aggregation was generated in the wells of the 24 hole plate. The aggregation was observed using an optical microscope and it was confirmed that there were a large number of microorganisms.

1 g of agarose (Invitrogen, UltraPure™ Agarose) was weighed out, and 200 mL of a CSiFF04 medium was put into a 500 mL conical flask. The CSiFF04 medium was subjected to an autoclave treatment for 10 minutes at 121° C., and approximately 20 mL of the CSiFF04 medium at a time was added to an Azunol Petri dish (1-8549-04, As One Corporation) in a clean bench before being cooled and hardened to produce agarose gel.

The solution containing the microalgae in the 24 hole plate was diluted, and the solution was made to adhere to a loop portion of a disposable stick (1-4633-12, As One Corporation) and was applied to the prepared agarose gel to prepare a Petri dish in which the microalgae were applied to the agarose gel.

The Petri dish was placed on a plant bioshelf for tissue culture and was cultured at 23° C. under continuous irradiation with 4000 lux light. After approximately 2 weeks, a green colony appeared on the agarose gel. The colony was adhered to a distal end of a sterilized bamboo skewer (1-5980-01, As One Corporation), and then, was suspended in the wells of the 24 hole plate into each of which 2 mL of the CSiFF04 medium was put. The 24 hole plate containing microalgae prepared in this manner was placed on a plant bioshelf for tissue culture and was cultured at 23° C. under continuous irradiation with 4000 lux light. After approximately 2 weeks, the aqueous solution in the wells exhibited a green color. A small amount of solution was collected from all of the wells, the microalgae were observed using an optical microscope, and it was concluded that purification had been performed in wells, in which it was considered that there was only a single microalga.

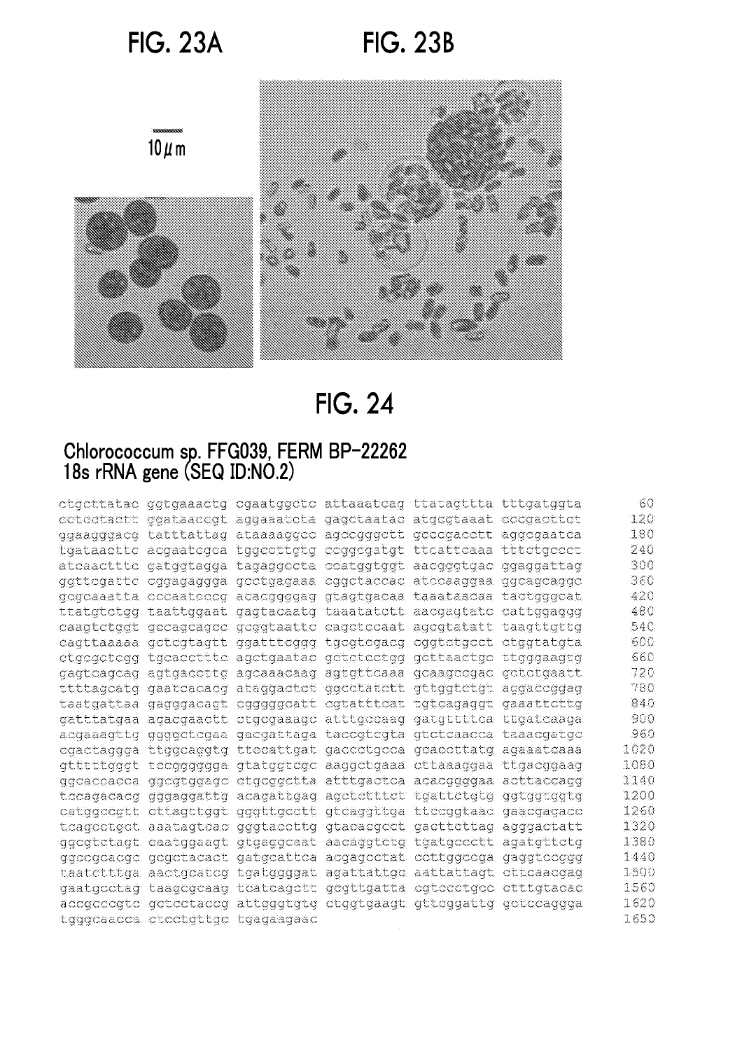

In addition, a microphotograph of FFG039 strains at magnification of 40 is shown in FIGS. 23A and 23B. FIG. 23A shows an ordinary state and FIG. 23B shows that the FFG039 strains proliferate by releasing a large number of zoospores.

[Morphological Properties]
All microalgae sink to the bottom surface if the microalgae are left for a while after performing a dispersion treatment.
If the microalgae are cultured for a while, microalgae floating on the liquid surface appear. Accordingly, microalgae are divided into microalgae having sunk to the bottom surface and microalgae floating on the liquid surface. If further culturing is performed continuously, a film-like structure appears on the liquid surface. If the culturing is performed further, a three-dimensional structure appears.
All the microalgae on the liquid surface and on the bottom surface have a spherical shape and have different size distributions.
The microalgae have cohesiveness and form a large colony.
The microalgae are green and the color thereof turns to yellow in accordance with the progress of the culturing.
There is little odor in collected substances and during the culturing.

[Culturing Properties]
During the cell proliferation, the cells proliferate through zoospores. A large number of zoospores are generated from one cell.
It is possible to perform photoautotrophic culture through photosynthesis.
Nitrogen, phosphorus, potassium, calcium, magnesium, sulfur, manganese, and iron are essential for proliferation. In addition, inclusion of zinc, cobalt, molybdenum, and boron makes the proliferation favorable. Addition of vitamins also promotes the proliferation.

[Physiological Properties]
Oil accumulates in an algal body at a maximum close to 40 wt % in terms of dry weight proportion.
In the oil, hydrocarbon compounds and fatty acids accumulate. The fatty acids produce palmitic acid, palmitoleic acid, oleic acid, vaccenic acid, linoleic acid, linolenic acid, and the like, and particularly, palmitic acid and oleic acid are main components. The hydrocarbon compounds produce decane, heptadecane, and the like.
FFG039 strains dyed with Nile Red were observed by a fluorescence microscope. Then, it was confirmed that there was oil which was colored with the Nile Red as a bright fluorescent light-emitting region in algal bodies in a fluorescent visual field. The oil can accumulate in a comparatively wider region within an alga body cell.

Identification of the FFG039 strains was further performed through the following method.

(Identification of FFG039 Strains of Microalgae)

The culture method of the FFG039 strains was as follows. 50 mL of a CSiFF04 medium was introduced into a conical flask with a 100 mL capacity, 0.5 mL of an FFG039 strain solution at a concentration of 1000×10⁴ cells/mL was added thereto, and shaking culture was performed under light irradiation for 14 days at 25° C.

In order to obtain a dry powder of the FFG039 strains, centrifugal operation was performed on 40 mL of the medium containing the FFG039 strains obtained as described above using a centrifuge (MX-300 (Tomy Seiko Co., Ltd.) for 10 minutes at a centrifugal force of 6000×g below 4° C. After removing a supernatant, the solid body was frozen together with the container using liquid nitrogen. Then, the total quantity of frozen solid body was transferred to a mortar which was chilled in advance using liquid nitrogen, and was ground using a pestle which was chilled in advance using liquid nitrogen.

The extraction of DNA from the microalgae was performed using DNeasy Plant Mini Kit (manufactured by Qiagen) according to the described manual. The purity and the amount of the extracted DNA were measured using e-spect (manufactured by Malcom Co., Ltd.). It was confirmed that the extracted DNA achieved the index of a purification degree which is A260 nm/A280 nm=1.8 or greater, and about 5 ng/μL of DNA was taken.

There was no problem in the purification degree of the extracted DNA, and thus, a sample for PCR was prepared by diluting the DNA 104 times in ultrapure water. An 18S rRNA gene region (rDNA region) was used as the sample for PCR. A cycle including 10 seconds at 98° C., 50 seconds at 60° C., and 10 seconds at 72° C. was performed 30 times for the PCR using a GeneAmp PCR System 9700 (manufactured by Applied Biosystems). An enzyme used herein was Prime Star Max (manufactured by Takara Bio Inc.). It was confirmed through 1% agarose electrophoresis that the obtained PCR product was a single band.

The purification of the PCR product was performed using a PCR purification kit (manufactured by Qiagen). The method was carried out through the method described in the manual. In order to check the purification degree and whether the PCR reaction was sufficiently performed, the purity and the amount of the DNA were measured using e-spect. It was determined that there was no problem since the measured purification degree was A260 nm/A280 nm=1.8 or greater.

Next, the purified substance was used as a template and a cycle sequence was performed using a BigDye Terminator v3.1 Cycle Sequencing kit (manufactured by Applied Biosystems). The manual was referred to for the condition. The base sequence of the obtained reactant was decoded using ABI PRISM 3100-Avant Genetic Analyzer (manufactured by Applied Biosystems).

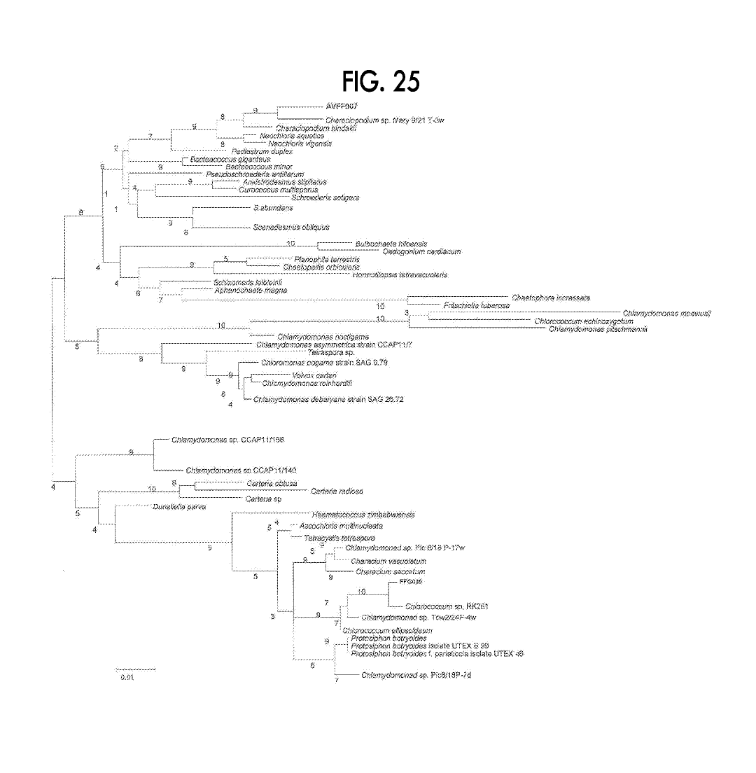

Identity analysis was performed using basic local alignment search tool (BLAST). The method thereof was as follows. BLAST searching for the above-described sequence was conducted on the whole base sequence information in the data of the National Center for Biotechnology Information (NCBI). A species of an organism having the highest identity was regarded as a closely related species of the FFG039 strains. Only the base sequence (1650 base, SEQ ID No: 1) which was set as a comparison target is shown in FIG. 24. Specifically, several bases at both the ends of the decoded base sequence were not set as a comparison target for the BLAST analysis, and thus, are not shown in FIG. 24. The upper left of the base sequence shown in FIG. 24 is a 5′-terminal and the lower right thereof is a 3′-terminal.

As a result of the identity analysis, the above-described sequence had the identity (that is, 99.94% identity) to a 1649 base on the FFG039 strain side among a Chlorococcum sp. RK261 strain side and 1650 bases on the Chlorococcum sp. RK261 strain side. Accordingly, the FFG039 strains were classified as microalgae closely related to the Chlorococcum sp. RK261 strains.

The systemic diagram obtained from the results of the above-described analysis is shown in FIG. 25. In a case where the name of the Chlorococcum is changed, similarly, it is regarded that the name of the FFG039 strains is also changed in the present invention.

In the present invention, it is possible to use strains having taxonomically the same properties as those of the FFG039 strains. The taxonomic properties of the FFG039 strains are shown below.

Taxonomic Properties of FFG039 Strains

### 1. Morphological Properties

The AVFF007 strains have a circular shape. When stationary culture is performed, a film-like structure is formed on the liquid surface. Oil is produced.

### 2. Culturing Properties (Culture Method)

(1) Medium: a CSiFF04 medium or a CSi improved medium (150 mg/L of Ca(NO₃)₂.4H₂O, 100 mg/L of KNO₃, 28.4 mg/L of K₂HPO₄, 22.2 mg/L of KH₂PO₄, 40 mg/L of MgSO₄.7H₂O, 588 ug/L of FeCl₃.6H₂O, 108 ug/L of MnCl₂.4H₂O, 66 ug/L of ZnSO₄.7H₂O, 12 ug/L of CoCl₂.6H₂O, 7.5 ug/L of Na₂MoO₄.2H₂O, 3 mg/L of Na₂EDTA.2H₂O, 0.1 ug/L of vitamin B12, 0.1 ug/L of Biotin, 10 ug/L of thiamine.HCl, pH 7.0)
(2) Culture temperature: culturing can be performed at 15° C. to 25° C.
(3) Culture period: for 2 to 4 weeks
(4) Culture method: stationary culture is suitable.
(5) Light-requiring properties: Necessary. Intensity of light: 4000 lux to 15000 lux. Bright and dark cycle: 12 hours for time for bright period and 12 hours for time for dark period.

As microalgae having the taxonomically same properties as those of the FFG039 strains, microalgae which belong to the genus Chlorococcum sp. and of which the 18S rRNA gene has sequence identity of at least 99.94% with polynucleotide formed of a base sequence of SEQ ID No: 2 are included.

[Floating Culture]

In the present invention, the culturing of microorganisms in a medium in a state of being dispersed is referred to as floating culture. In the present invention, the culturing on the liquid surface is not referred to as floating culture. The floating culture is used in accordance with the purpose in a pre-culture process.

[Liquid Surface-floating Culture]

In the present invention, a culture method for culturing microorganisms on the liquid surface is referred to as liquid surface-floating culture. Even if microorganisms exist on the bottom surface of a culture vessel, on a side surface thereof, in the middle of a medium, or the like at the same time, the culture method is also referred to as liquid surface-floating culture in a case where the main purpose of the culture method is to perform culturing on the liquid surface. In addition, in a case where a biofilm is formed on the liquid surface, a large amount of bubble exists on the liquid surface together with a biofilm. Therefore, there is a case where the position of the liquid surface is not always clear, or a case where the biofilm is slightly sunk under the liquid surface due to its own weight. Such a case is included as well as a complete liquid surface when the top of the liquid surface is mentioned in the present invention. However, a culture method in which microorganisms are cultured in only either or only both of the middle of a solution or the bottom surface of a culture vessel is not included in the liquid surface-floating culture.

The liquid surface in the present invention refers to a typical liquid surface of a liquid medium to be described below, and in general, refers to an interface between the liquid medium and the air. In addition, in a case where water is a main component, the liquid surface refers to a water surface.

In addition, when liquid surface-floating culture is performed in the present invention, in some cases, a phenomenon in which a pleat-like structure enters the middle of a liquid from a film-like structure or a three-dimensional structure on the liquid surface can be seen. In the present invention, culturing in such a situation is also included in the liquid surface-floating culture.

Seed microorganisms for performing liquid surface-floating culture may be subjected to suspension treatment, and then, may be added to a culture vessel. Alternately, after adding seed microorganisms to the culture vessel, the seed microorganisms may be stirred in order to accelerate mixing of the seed microorganisms and a liquid medium. In addition, a biofilm of microorganisms may be added to the surface of water of a culture vessel to start culturing in a state in which the biofilm is made to float. In addition, the biofilm of microorganisms may be broken such that separation of the biofilm of microorganisms from the surface of water of the biofilm of microorganisms minimally occurs after the floating, and may be stirred so as to be dispersed on the liquid surface of the culture vessel.

[Adhesive Culture]

The adhesive culture referred to in the present invention is to perform culturing in a state in which microorganisms are adhered on the surface of a substrate or the wall surface of a culture vessel (for example, the bottom surface or a side surface of a culture vessel). The primary culturing process of the present invention is a type of adhesive culture.

[Wall Surface Culture]

The wall surface culture referred to in the present invention is a culture method, which is performed by installing a structure of a substrate and a water-absorbent polymer gel at an angle greater than or equal to 45 degrees with respect to the ground, and is a culture method which is performed in a state in which microorganisms are adhered. The wall surface culture includes vertical culture. In a case of performing the wall surface culture, it is preferable to use a support substrate.

When disposing the structure, it is preferable to use an instrument for fixing the structure. In addition, a plurality of structures may be installed and the culturing may be started at the same time. With the installation of the plurality of structures, it is possible to effectively use the area required for the culturing.

The interval for installing the structures can be arbitrarily determined, and is preferably greater than or equal to 5 mm, more preferably greater than or equal to 1 cm, and most preferably greater than or equal to 10 cm. If the interval between the structures is greater than or equal to 5 mm, it is possible to make light reach the vicinity of bottom portions of the structures as well. The interval for installing the structures is generally less than or equal to 1000 cm.

The height of structures at the time of vertically disposing the structures with respect to the ground can also be determined in accordance with the purpose of culturing. All of the structures may be installed to have the same height as each other, or may be installed to have different heights from each other. Accordingly, in some cases, it is possible to efficiently perform culturing when light is obliquely emitted.

[Vertical Culture]

The vertical culture referred to in the present invention is a culture method which is performed by installing a structure formed of a substrate and a water-absorbent polymer gel at an angle greater than or equal to 70 degrees with respect to the ground, and is a culture method which is performed in a state in which microorganisms are adhered. A form of the wall surface culture is the vertical culture.

[Horizontal Culture]

The horizontal culture referred to in the present invention is a culture method which is performed by installing a structure formed of a substrate and a water-absorbent polymer gel at an angle of less than 45 degrees with respect to the ground.

[Double-surface Culture]

The double-surface culture referred to in the present invention is a culture method for performing culturing using two surfaces, out of the surfaces which a water-absorbent polymer gel has. The double-surface culture is mainly a culture method which is performed in the wall surface culture and in which a support substrate is preferably used. In the case of using a support substrate, in some cases, water-absorbent polymer gel layers are respectively installed on both sides of the support substrate as shown in FIG. 5. In this case, it is possible to use one surface of the water-absorbent polymer gel layer on one side of the support substrate and one surface of the water-absorbent polymer gel layer on the other side of the support substrate, for culturing. Moreover, the case of performing culturing using these two surfaces is also included in the double-surface culture referred to in the present invention.

[Pre-Culture Process]

The pre-culture process of the present invention is a process of increasing the number of microorganisms until the primary culture can be performed by causing microorganisms for storage, which have been obtained after the completion of a purification process, to proliferate. Any well-known culture method can be selected as the pre-culture process. For example, a dispersion culture method or an adhesion culture method, or liquid surface-floating culture or the like which has been developed by the present inventors can be performed. In addition, pre-culture process may be performed several times in order for microorganisms to proliferate until the microorganisms reach a scale in which primary culture can be performed.

In addition, culturing can be performed in either of an indoor place or an outdoor place using a culture vessel having a surface area less than or equal to 1 cm² to 1 m², but culturing indoors is preferable.

[Primary Culture Process]

The primary culture process is a culture process which is performed after the pre-culture process is performed and immediately before the final collecting process is performed. The primary culturing process may be performed plural times.

In addition, a culture vessel having a surface area greater than or equal to 100 cm² is generally used. It is possible to perform the culturing in either of an indoor place or an outdoor place, but it is preferable to perform the culturing outdoors.

[Seed Microorganisms]

Seed microorganisms of the present invention refer to microorganisms which are used when starting the above-described pre-culture process or primary culture process, and become a base for culturing microorganisms in the pre-culture process or the primary culture process. Furthermore, the seed microorganisms are not limited to microorganisms obtained through the pre-culturing process, and it is also possible to use microorganisms obtained through the primary culturing process, and a part of a final collected substance obtained in the collecting process.

In addition, in a case where culturing is started again using microorganisms remaining on a substrate or a water-absorbent polymer gel after the collecting process, it is possible to handle these microorganisms as seed microorganisms.

In a case where the microorganisms are microalgae, in some cases, the microorganisms are called seed algae in the present invention.

[Suspension Treatment]

In the present invention, a sample of microorganisms subjected to suspension treatment may be used. This is because, with the suspension treatment, the microorganisms in a solution are uniformized, the distribution of the microorganisms on a water-absorbent polymer gel or a substrate is uniformized, and the film thickness after culturing is uniformized, and as a result, in some cases, the quantity of microorganisms per culturing area increases. Any well-known method can be used for the suspension treatment, and examples thereof include gentle treatment such as treatment of pipetting or shaking a solution of microorganisms put into a container by hand and treatment using a stirrer chip or a stirring rod; strong treatment such as ultrasonic treatment or high speed-shaking treatment; and a method of using a substance such as an enzyme decomposing an adhesion substance such as an intracellular matrix.

However, in a case of microorganisms which do not exhibit cohesiveness, this treatment process is not required. In addition, in the case of microorganisms obtained through liquid surface-floating culture in FIG. 3A to FIG. 3F, this treatment process is not required except for a case of coating the surface of a substrate or a water-absorbent polymer gel.

[Coating with Microorganisms]

Coating with microorganisms refers to treatment of making microorganisms exist at least any one of the surface of a water-absorbent polymer gel and the surface of a substrate, and any well-known method may be used for the method. Examples thereof include a method for adding a solution containing microorganisms to the above-described surface, and then, coating the surface with the solution using a spreading bar or the like; a method for immersing the above-described surface in a microorganism suspension liquid and making microorganisms adhere to the surface; and a method for transferring a biofilm of microorganisms formed on the liquid surface, to the above-described surface. It is preferable to perform suspension treatment on the solution containing microorganisms since there are many cases where it is possible to uniformly coat the surface with microorganisms.

In addition, the coating amount of microorganisms when starting culturing is preferably 0.001 μg/cm² to 1 mg/cm², more preferably 0.1 μg/cm² to 0.1 mg/cm², and most preferably 1 μg/cm² to 10 μg/cm². If the coating amount of microorganisms is greater than or equal to 0.1 μg/cm², it is possible to increase the proportion of the quantity of microorganisms after the completion of culturing with respect to the amount of microorganisms at the time of starting the culturing, within a short period of time, which is preferable.

In addition, a plurality of aggregations of microorganisms may exist within a culture region.

[Preparation of Microorganism-Adhered Substrate Using Method of Transferring Biofilm on Liquid Surface]

The transferring method is a method for transferring a biofilm (a film-like structure or a three-dimensional structure) of microorganisms, which has been formed of microorganisms on the liquid surface as shown in FIG. 3D to FIG. 3E, to a substrate and is a type of adhesion which is performed without substantial proliferation.

The substrate is gently inserted with respect to the liquid surface so as to be parallel to or at an angle close to the liquid surface, and the biofilm of microorganisms on the liquid surface is adhered to surface of the substrate. When performing the insertion, the substrate is slightly obliquely inserted with respect to the liquid surface, and is then finally made to be parallel to the liquid surface. Then, it is possible to perform adhesion of a large amount of biofilm with a smaller number of times of transferring, which is preferable. The transferring may be performed plural times in terms of improving the transferring rate.

In the transferring, a substrate may be brought into contact with the entirety of the liquid surface of a culture vessel, or may be partially brought into contact with the liquid surface thereof. In the transferring of a biofilm, in a case of transferring a part of the biofilm and using a plurality of substrates, it is preferable to bring the plurality of substrates into contact with the liquid surface, and then to lift the biofilm-adhered substrates from the liquid surface. The reason for this is as follows. After inserting one sheet of a substrate into the liquid surface, a region in which there is no biofilm appears at the same time when the substrate is lifted from the liquid surface. There is a possibility that the structure of the biofilm is collapsed by movement of the liquid surface caused by the lifting, and therefore, the collapsed biofilm will move from a region where there is a biofilm to a region where there is no biofilm. Accordingly, when the transferring is performed using a new substrate, in some cases, the transferring is performed on the region where there is a biofilm and a region where there is no biofilm, at the same time. Therefore, in this case, the proliferation efficiency in a primary culturing process is deteriorated.

[Coating of Water-Absorbent Polymer Gel with Substrate]

Any well-known method may be used as long as it is possible to coat a water-absorbent polymer gel with a substrate.

In some cases, a gas phase is generated between a water-absorbent polymer gel and a substrate through coating. Culturing may be performed while the gas phase is left intact. However, in a case where there are microorganisms on the substrate side, it is preferable to remove the gas phase as possible since the gas phase causes various problems such as a decrease in the proliferation rate due to drying of microorganisms on the substrate side of the phase portion, decrease in properties of a biofilm of microorganisms being detached from a substrate due to dying out or drying of microorganisms, or the like.

The coating of a water-absorbent polymer gel with a substrate may be performed immediately after coating either or both of the water-absorbent polymer gel and the substrate, with microorganisms. However, the coating of the water-absorbent polymer gel with the substrate may also be performed after culturing is performed for a certain time.

[Culture Vessel]

Any well-known shape can be used as the shape of a culture vessel (culture pond) as long as the culture vessel can hold a water-absorbent polymer gel. As the culture vessel, any of an open type and a closed type can be used. However, the closed-type culture vessel is preferably used in order to prevent diffusion of carbon dioxide to the outside of the culture vessel when the concentration of carbon dioxide which is higher than that in the air is used. By using the closed-type culture vessel, it is possible to prevent microorganisms other than microorganisms for culturing, or debris from being mixed in; to suppress evaporation of a medium, and to minimize an influence of wind on the structure. However, in a case of performing commercial production, culturing in an open system is preferable from the viewpoint that the construction costs are reduced.

[Substrate]

The substrate referred to in the present invention is a solid substance used as 4 in FIGS. 1A to 1H, 4 in FIGS. 2A and 2B, 4 in FIGS. 3A to 3F, 4 in FIGS. 4A to 4L, and 2 in FIG. 5, and mainly has at least a function selected from a function of preventing a water-absorbent polymer gel or microorganisms from being dried; a function of maintaining the shape of a water-absorbent polymer gel; a function of transferring a biofilm of microorganisms on the liquid surface; a function of mediating going in and out of a gaseous substance which is required or not required for metabolism of microorganisms, and a function of preventing entrance of non-target microorganisms.

As the shape of the substrate, any shape such as a film shape, a plate shape, a fibrous shape, a porous shape, a convex shape, and a wavy shape may be used. In terms of ease of transfer, ease of detaching microorganisms from a substrate, and a high ability of supporting a water-absorbent polymer gel, a film shape or a plate shape is preferable.

In addition, it is possible to use a substrate bored with a hole, that is, a substrate having a penetrating structure. In a case of microorganisms which release gas in accordance with the progress of culturing, since microorganisms are cultured between a water-absorbent polymer gel and a substrate in the present invention, it is difficult for the gas to diffuse in the air in this region. Such a problem barely occurs in a case of using a substrate with good gas permeability. In a case of using a substrate with inferior gas permeability or microorganisms which generate a large amount of gas, it is possible to bore a hole, through which gas diffuses outside a culture vessel, on the surface of a substrate. The number or the interval of holes are not particularly limited, as long as gas can diffuse outside a culture vessel and the number or the interval of holes remarkably does not affect culturing of microorganisms.

In a case where gas remains between a water-absorbent polymer gel and a substrate, it is difficult to secure moisture for microorganisms proliferating (particularly in a case where there are a large amount of microorganism on the substrate side), and there is a possibility that the proliferation rate will decrease. In addition, in a case where such bubbles are generated, there is a possibility that microorganisms strongly adsorb onto a substrate, thereby causing a problem when detaching microorganisms from the substrate. For this reason, it is important to install a penetrating structure in a substrate and to make the generated gas diffuse outside a culture vessel.

[Support Substrate]

The support substrate in the present invention is a type of a substrate and is a substrate which is used as 2 in FIGS. 1A to 1H, 4 in FIGS. 4A to 4L, and 2 in FIG. 5, and is mainly used in order to maintain a structure of a water-absorbent polymer gel. In general, the substrate has a more increased strength.

[Unevenness on Surface of Substrate]

In the present invention, it is also possible to form unevenness on the surface of a substrate. This is because, in some cases, the unevenness structure makes a gaseous substance easily diffuse through a region between the substrate and a water-absorbent polymer gel layer.

[Material]

The materials of the culture vessel, the substrate, and the support substrate that can be used in the present invention are not particularly limited, and well-known materials can be used. For example, it is possible to use a material formed of an organic polymer compound, an inorganic compound, metal, or a composite thereof. In addition, it is also possible to use a mixture thereof

Polyethylene derivatives, polyvinyl chloride derivatives, polyester derivatives, polyamide derivatives, polystyrene derivatives, polypropylene derivatives, polyacrylic derivatives, polyethylene terephthalate derivatives, polybutylene terephthalate derivatives, nylon derivatives, polyethylene naphthalate derivatives, polycarbonate derivatives, polyvinylidene chloride derivatives, polyacrylonitrile derivatives, polyvinyl alcohol derivatives, polyethersulfone derivatives, polyarylate derivatives, allyl diglycol carbonate derivatives, ethylene-vinyl acetate copolymer derivatives, fluorine resin derivatives, polylactic acid derivatives, acrylic resin derivatives, ethylene-vinyl alcohol copolymers, ethylene-methacrylic acid copolymers, and the like can be used as the organic polymer compound.

Glass, ceramics, concrete, and the like can be used as the inorganic compound.

Alloys such as iron, aluminum, copper, or stainless steel can be used as a metallic compound.

Among the above, it is preferable that a part of the material of the substrate or the culture vessel is formed of at least one selected from glass, polyethylene, polypropylene, nylon, polystyrene, vinyl chloride, and polyester.

In addition, the materials of the culture vessel, the substrate, and the support substrate may be the same as each other or different from each other.

In addition, in a case of using a closed-type culture vessel, the light receiving surface may be made of a material through which light is transmitted, and a transparent material is more preferable. In addition, in a case of performing vertical culture, it is preferable that the substrate or the support substrate is a transparent material.

[Water-Absorbent Polymer]

The water-absorbent polymer in the present invention is a polymer which is excellent in water absorbency and can hold a large amount of moisture (including a medium), and is a polymer which has properties in which it is difficult to separate water from the polymer, even if pressure is applied thereto after the absorption of water. In general, the water-absorbent polymer has a gel-like foam by forming a cross-linking structure (in the present invention, also referred to as a mesh structure, a network structure, or the like) and taking a water molecule therein.

Water absorbing capacity of the water-absorbent polymer is preferably 2 times to 10,000 times heavier than its own weight, more preferably 10 times to 1,000 times heavier than its own weight, and particularly preferably 50 times to 500 times heavier than its own weight. Here, the water absorbing capacity indicates a water absorbing capacity obtained by measuring the weight of water absorbed with respect to the weight of polymer dried, using pure water. However, the subject from which the water-absorbent polymer absorbs water in the present invention is not limited to pure water, and a medium, water, or the like to be described below is intended. In general, in a case of using a water solution, which contains salts, instead of pure water, the water absorbing capacity is deteriorated.

As a monomer constituting the above-described water-absorbent polymer, there is no particular limitation as long as the water-absorbent polymer has the above-described water absorbing capacity after polymerization, and any monomer can be appropriately selected in accordance with the purpose. Examples thereof include acrylic acid, acrylic acid derivatives, vinyl acetate, carboxymethyl cellulose, ethylene, a methacrylate derivative, pyrrolidone, aliphatic glycol, propylene, cellulose derivatives, and amino acid. Examples of the above-described acrylic acid derivatives include methacrylic acid and esters thereof, calcium salts and sodium salts, hydroxyethyl methacrylate, hydroxypropyl methacrylate, 2-hydroxybutyl acrylate, dimethylaminoethyl methacrylate, acrylic acid hydroxyalkyl esters, acrylamide and derivatives thereof (N-methylol acrylamide and alkyl ether compounds thereof, or the like), acrylic acid derivatives having an oxirane group (glycidyl acrylate, methacrylonitrile, or the like), acrylonitrile, methyl acrylate, ethyl acrylate, propyl acrylate, n-butyl acrylate, isobutyl acrylate, t-butyl acrylate, hexyl acrylate, octyl acrylate, 2-ethylhexyl acrylate, lauryl acrylate, stearyl acrylate, acetyl acrylate, dodecyl acrylate, methyl methacrylate, ethyl methacrylate, propyl methacrylate, n-butyl methacrylate, isobutyl methacrylate, t-butyl methacrylate, hexyl methacrylate, octyl methacrylate, 2-ethylhexyl methacrylate, lauryl methacrylate, stearyl methacrylate, acetyl methacrylate, and dodecyl methacrylate.

As the above-described water-absorbent polymer, in addition to a polymer compound used when polymerizing the above-described monomer, there is no particular limitation as long as the water-absorbent polymer has the above-described water absorbing capacity, and any water-absorbent polymer can be appropriately selected in accordance with the purpose. Examples thereof include polyacrylamide, polyvinyl alcohol, carboxymethyl cellulose, ethylene-vinyl alcohol copolymers, poly-hydroxyethyl methacrylate, poly-α-hydroxy polyvinyl alcohol, polyacrylic acid, poly-α-hydroxy acrylate, polyvinyl pyrrolidone, polyalkylene glycol such as polyethylene glycol, polypropylene glycol; sulfonated substances thereof; sodium alginate; starch; silk fibroin; silk sericin; gelatin; various proteins; and polysaccharides. These may be used alone or in a combination of two or more thereof.

In addition, it is possible to use an extracellular matrix, which is produced by microorganisms, as a water-absorbent polymer. Microorganisms may exist in the extracellular matrix, or only the extracellular matrix may be taken out and used. Furthermore, in accordance with the extracellular matrix contains microorganisms, the microorganisms may be used while being alive, or may be used while being dead.

As the molecular weight of the above-described water-absorbent polymer, there is no particular limitation and a molecular weight can be appropriately selected in accordance with the purpose. The mass average molecular weight is preferably 1,000 to 10,000,000, more preferably 2,000 to 1,000,000, and particularly preferably 5,000 to 100,000. If the above-described mass average molecular weight is greater than or equal to 1,000, the structure of a water-absorbent polymer gel is stabilized, and if the above-described mass average molecular weight is less than or equal to 10,000,000, polymerization is easily performed. In contrast, the mass average molecular weight being from 5,000 to 100,000 is preferably in terms of stability of the water-absorbent polymer gel.

The above-described water-absorbent polymer gel may be or may not be cross-linked. However, the above-described water-absorbent polymer gel is preferably cross-linked from the viewpoint of minimizing the influence on a structure in a case where microorganisms are detached from the surface, the viewpoint of repetitive use, the viewpoint of necessity of holding the form of the water-absorbent polymer gel during vertical culture, or the like.

The method for cross-linking a water-absorbent polymer gel is not particularly limited, and any well-known method can be appropriately selected. Examples thereof include a method of using a cross-linking agent, a method of using a radical initiator, a cross-linking method through heating, and a method of using an electron ray, an ultraviolet ray, a radial ray, or the like. Among these, a method of using a cross-linking agent and a method of using an ultraviolet ray are preferable from the viewpoint of simplicity, high cross-linking efficiency, and the safety.

As the above-described water-absorbent polymer gel, a copolymer may be used. Use of the copolymer has an advantage in that a cross-linking reaction becomes easy.

The amount of the above-described water-absorbent polymer with respect to a support substrate is not particularly limited and can be appropriately selected in accordance with the purpose. The amount thereof is, by mass of dry powders, preferably 1 μg/cm² to 100 g/cm² per the area of the above-described substrate, more preferably 100 μg/cm² to 1 μg/cm² per the area of the above-described substrate, and particularly preferably 1 mg/cm² to 100 mg/cm² per the area of the above-described substrate. If the amount of the above-described water-absorbent polymer is greater than or equal to 1 μg/cm², the structure of the water-absorbent polymer gel is stabilized, and if the amount thereof is less than or equal to 100 g/cm², the moisture can be sufficiently accumulated.

The thickness of water-absorbent polymer gel is preferably 1 mm to 100 cm, more preferably 5 mm to 20 cm, and particularly preferably 1 cm to 5 cm. If the thickness of water-absorbent polymer gel is greater than or equal to 1 mm, it is possible to sufficiently maintain moisture, and if the thickness of water-absorbent polymer gel is less than or equal to 100 cm, it is possible to maintain the faint of the gel layer.

[Medium (Liquid Medium)]

In the present invention, as a medium used for culturing and a medium with which the water-absorbent polymer compound is impregnated, any well-known medium (liquid medium) can be used, as long as it is possible to culture microorganisms. Examples of well-known media include an AF-6 medium, an Allen medium, a BBM medium, a C medium, a CA medium, a CAM medium, a CB medium, a CC medium, a CHU medium, a CSi medium, a CT medium, a CYT medium, a D medium, an ESM medium, an f/2 medium, an HUT medium, an M-11 medium, an MA medium, an MAF-6 medium, an MF medium, an MDM medium, an MG medium, an MGM medium, an MKM medium, an MNK medium, an MW medium, a P35 medium, a URO medium, a VT medium, a VTAC medium, a VTYT medium, a W medium, a WESM medium, an SW medium, and an SOT medium. Among these, freshwater media are an AF-6 medium, an Allen medium, a BBM medium, a C medium, a CA medium, a CAM medium, a CB medium, a CC medium, a CHU medium, a CSi medium, a CT medium, a CYT medium, a D medium, an HUT medium, an M-11 medium, an MA medium, an MAF-6 medium, an MDM medium, an MG medium, an MGM medium, an MW medium, a P35 medium, a URO medium, a VT medium, a VTAC medium, a VTYT medium, a W medium, an SW medium, and an SOT medium. As media for culturing the above-described AVFF007 strains, a C medium, a CSi medium, and a CHU medium, and a mixture of these media are preferable. It is desirable to select the medium in accordance with the types of microorganisms to be cultured. In addition, a medium may be contained in a water-absorbent polymer gel.

The media may be subjected to ultraviolet ray sterilization, autoclave sterilization, or filter sterilization, or may not be sterilized.

Different media may be used as media in the pre-culture process and the primary culture process. In addition, a medium may be changed to another medium during the culture processes.

[Reuse of Water-absorbent Polymer Gel and Substrate]

In the present invention, water-absorbent polymer gel can be limited to be used once. However, the water-absorbent polymer gel is preferably reused from the viewpoints of effective use of resources and cost reduction.

In this case, after detaching a biofilm of microorganisms from the surface of a substrate or water-absorbent polymer gel after culturing, the water-absorbent polymer gel may be coated with the substrate to start culturing. That is, it is impossible to completely detach microorganisms, and therefore, this is a method for starting the culturing by having microorganisms remaining on the surface as seed microorganisms.

Furthermore, after coating the top of water-absorbent polymer gel or the top of the substrate with a suspension liquid of microorganisms, both of the water-absorbent polymer gel or the substrate may be brought into contact with each other.

The water-absorbent polymer gel may be coated with the substrate after adding a medium to the water-absorbent polymer gel. That is, culturing may be started after newly adding nutrient components for proliferation to the water-absorbent polymer gel. In this case, the culturing may be started without newly preparing seed microorganisms. Furthermore, after performing drying process for reducing the moisture content of water-absorbent polymer gel, a medium may be added to the water-absorbent polymer gel, which may then be coated with the substrate. After adding a medium to the water-absorbent polymer gel, in a case where a liquid medium remains on the surface thereof, the water-absorbent polymer gel may be coated with a substrate after drying the water-absorbent polymer gel to the extent that no liquid medium exists on the surface thereof. Even in this case, the culturing may be started without newly preparing seed microorganisms. In addition, as the substrate, a new substrate may be used, or a medium which has been used for culturing once may be used. In addition, a medium having components the same as or different from that when preparing water-absorbent polymer gel may be used as the medium, or the concentration thereof may be changed. It is more preferable to add a medium with a higher concentration from the viewpoint of avoiding a problem of a limit value of water absorbency with respect to water-absorbent polymer gel. In addition, in these processes, after coating an agar medium with microorganisms, the microorganisms may be coated with a substrate. Alternately, a water-absorbent polymer gel may be coated with a microorganism-adhered substrate after making microorganisms adhere to the substrate. In addition, at least one of a water-absorbent polymer gel and a substrate after being used may be used after being washed with distilled water or a medium.

In a case of culturing different microorganisms, it is desirable to reuse a water-absorbent polymer gel or a substrate after sufficient examination.

The substrate or the water-absorbent polymer gel may be used after subjecting the substrate or the water-absorbent polymer gel to sterilization treatment. Particularly, in a case of changing the types of microorganisms, the above-described method may be considered examined.

[Carbon Dioxide]

In a case of using microalgae as microorganisms, it is necessary to supply carbon dioxide in order to make a large quantity of proliferate.

In a case of performing dispersion culture in a pre-culture process, carbon dioxide may be supplied to a medium through bubbling which is a conventional method. However, in a case of using liquid surface-floating culture, it is preferable to supply carbon dioxide from a gas phase. This is because there is a possibility that the structure of a microalgal biofilm on the liquid surface may be destroyed, unevenness in the quantity of algal bodies may occur, or the efficiency of collecting a biofilm on a substrate through a collecting process may be deteriorated, and therefore, the quantity of algal bodies collected may decrease.

In the primary culturing process, since the culturing is performed on water-absorbent polymer gel, it is, in principle, impossible to supply carbon dioxide through bubbling. Therefore, carbon dioxide is supplied from a gas phase. Regarding the substrate, it is preferable to bore a hole penetrating the substrate in at least one or more sites. In addition, it is preferable to use a plurality of substrates with a small area. It is also possible to use a substrate having carbon dioxide permeability. As such a substrate, it is possible to use a silicone rubber sheet or the like.

In the present invention, it is possible to use carbon dioxide in the air, but it is also possible to use carbon dioxide having a higher concentration than that in the air. In this case, it is desirable to perform culturing in a closed-type culture vessel or in a culture vessel which is covered with a coating material such as an agricultural film, in order to prevent the loss of carbon dioxide due to diffusion. The concentration of carbon dioxide in this case is not particularly limited as long as it is possible to achieve the effect of the present invention, but is preferably greater than or equal to the concentration of carbon dioxide in the air and less than 20 volume %, more preferably 0.01 volume % to 15 volume %, and still more preferably 0.1 volume % to 10 volume %. In addition, carbon dioxide may be discharged using a combustion device. In addition, carbon dioxide may also be generated using a reagent.

[Light Source and Amount of Light]

As light sources that can be used in the present invention, any light source can be used. However, it is possible to use sunlight, LED light, a fluorescent lamp, an incandescent lamp, xenon lamp light, a halogen lamp, and the like. Among these, it is preferable to use sunlight as natural energy, an LED having a good luminous efficiency, or a fluorescent lamp that can be simply used.

The amount of light is preferably 100 lux to 1000000 lux and more preferably 300 lux to 500000 lux. The most preferable amount of light is 1000 lux to 200000 lux. If the amount of light is greater than or equal to 1000 lux, it is possible to culture microalgae, and if the amount of light is less than or equal to 200000 lux, there is a little adverse effect on culturing due to photolesion.

Light may be radiated through any method such as continuous irradiation, and repetition of irradiation and non-irradiation at a constant time interval, but it is preferable that light be turned on and off at a time interval of 12 hours.

The wavelength of light is not limited, and any wavelength can be used as long as the wavelength is a wavelength at which photosynthesis can be performed. A preferred wavelength is a wavelength of sunlight or a wavelength similar to that of sunlight. An example in which the growth rate of photosynthetic organisms is improved by radiating a single wavelength has been reported, and even in the present invention, it is also possible to use such an irradiation method.

[Other Culture Conditions]

In the present invention, the pH of a liquid medium used in the pre-culturing process, a liquid medium with which a water-absorbent polymer gel is impregnated, and a liquid medium (hereinafter, the liquid medium is also referred to as a culture solution) used in a case of reusing a water-absorbent polymer gel, which are used in a pre-culture process is preferably within a range of 1 to 13, more preferably within a range of 3 to 11, still more preferably within a range of 5 to 9, and most preferably within a range of 6 to 8.

In addition, it is preferable to select the pH of the medium in accordance with the types of microorganisms since a preferred pH is changed in accordance with the types of microorganisms. The pH of the liquid medium refers to the pH when starting the culturing. In addition, in some cases, the pH during a culture process is changed accompanying the culturing, and therefore, the pH during the culture process may be changed.

In the present invention, it is possible to add a substance, which has a buffer action, to a medium for maintaining a constant pH in the medium. Accordingly, in some cases, it is possible to suppress a problem in which the pH in a medium is changed in accordance with the progress of culturing of microorganisms, or to suppress the phenomenon in which the pH is changed due to supply of carbon dioxide to the medium. As the substance having a buffer action, it is possible to use a well known substance. The use thereof is not limited, but it is possible to suitably use 4-(2-hydroxyethyl)-1-piperazineethanesulfonic acid (HEPES), a sodium phosphate buffer solution, a potassium phosphate buffer solution, or the like. The concentrations or the kinds of buffer substances can be determined in accordance with the types or the culture environments of the microorganisms.

The culture temperature can be selected in accordance with the types of microorganisms and is not particularly limited. However, the culture temperature is preferably 0° C. to 90° C., more preferably 15° C. to 50° C., and most preferably 20° C. to 40° C. If the culture temperature is greater than or equal to 20° C. and less than 40° C., it is possible to suitably culture microorganisms.

If the lower limit quantity of input microorganisms, that is, the quantity of microorganisms used when starting culturing is one within a range of culturing, microorganisms can proliferate for as long as the time that is given, and therefore, there is no particular restriction. However, the lower limit input microalgae quantity thereof is preferably greater than or equal to 1 cell/cm², more preferably greater than or equal to 1000 cells/cm², and still more preferably greater than or equal to 1×10⁴ cells/cm². Regarding the upper limit quantity of input microorganisms, microorganisms can proliferate at any high concentration, and therefore, there is no particular restriction. However, the upper limit quantity of input microorganisms is preferably lower than or equal to 1×10⁹ cells/cm², more preferably lower than or equal to 1×10⁸ cells/cm², and still more preferably lower than or equal to 5×10⁷ cells/cm² from the viewpoint that, when the concentration thereof is higher than a certain concentration, the ratio of the number of microorganisms after the proliferation to the number of input microorganisms decreases.

The pre-culture period and the primary culture period in the present invention can be selected in accordance with the types of microalgae and is not particularly limited. However, the pre-culture period and the primary culture period is preferably 1 day to 300 days, more preferably 3 days to 100 days, and still more preferably 7 days to 50 days.

The water depth of a liquid medium to be used in liquid surface-floating culture is not particularly limited, but the water depth is preferably shallow. This is because the amount of water used is small and the handling efficiency improves. The water depth thereof is preferably greater than or equal to 0.4 cm, more preferably 1 cm to 10 cm, still more preferably 2 cm to 1 m, and most preferably 4 cm to 30 cm. If the water depth thereof is greater than or equal to 0.4 cm, it is possible to form a biofilm. If the water depth thereof is less than or equal to 10 m, it becomes easy to handle the liquid medium. If the water depth is 4.0 cm to 30 cm, the influence caused by evaporation of moisture is minimized, and it is easy to handle a medium or a solution containing microalgae.

[Size and Proliferation Rate of Biofilm of Microorganisms which is Grown in Region Surrounded between Water-absorbent Polymer Gel and Substrate]

The size of a biofilm of microorganisms is preferably greater than or equal to 0.1 cm², more preferably greater than or equal to 1 cm², still more preferably greater than or equal to 10 cm², and most preferably the same as the area of a substrate which is in contact with a water-absorbent polymer gel layer. If the size thereof is greater than or equal to 0.1 cm², it is possible to increase the ratio of the quantity of microorganisms after the completion of culturing to the quantity of microorganisms when starting culturing within a short period of time, which is preferable. In addition, a plurality of biofilms of microorganisms may exist within a culture region. Even in liquid surface-floating culture, culturing of microorganisms at sizes of these biofilms is performed within a preferred range. In this case, the area of a substrate which is in contact with the above-described water-absorbent polymer gel layer becomes the surface area of the liquid surface of a culture vessel.

The thickness of a biofilm of microorganisms is preferably within a range of 1 μm to 10 cm, more preferably within a range of 10 μm to 5 cm, and most preferably within a range of 100 μm to 1 cm. If the thickness thereof is greater than or equal to 1 μm, a sufficient amount of final collected substance can be obtained, and if the thickness thereof is less than or equal to 10 cm, it is possible to reduce the death rate of microorganisms during culturing by supplying a sufficient amount of moisture to a film layer, and to deliver light, nutrients, such as carbon dioxide, which are required for culturing, or energy.

In addition, microorganisms according to the present invention preferably have a high proliferation rate on the liquid surface. The proliferation rate (that is, an average proliferation rate per day during a period of a logarithmic proliferation phase) of microorganisms in the logarithmic proliferation phase is preferably greater than or equal to 0.1 g/m²/day by dry weight, more preferably greater than or equal to 0.5 g/m²/day by dry weight, still more preferably greater than or equal to 1 g/m²/day by dry weight, and most preferably greater than or equal to 3 g/m²/day by dry weight. The proliferation rate of microalgae in the logarithmic proliferation phase is generally less than or equal to 1000 g/m²/day by dry weight. Even in liquid surface-floating culture, culturing of microorganisms at these proliferation rates is performed within a preferred range.

In addition, in a biofilm of microorganisms which is formed between water-absorbent polymer gel and a substrate according to the present invention, the weight of dry microorganisms per unit area of water-absorbent polymer gel or a substrate is preferably 1 μg/cm² to 100 g/cm², more preferably 50 μg/cm² to 10 g/cm², and most preferably 0.5 mg/cm² to 1 g/cm². This is because, if the weight thereof is greater than or equal to 1 μg/cm², it is possible to sufficiently obtain final collected substance, and if the weight thereof is less than or equal to 100 g/cm², it is possible to reduce the death rate of microorganisms during culturing by supplying a sufficient amount of moisture to a biofilm layer, and to deliver light, nutrients such as carbon dioxide which are required for culturing, or energy.

[Collection]

It is possible to collect a biofilm in a state of being partially covered with a region between water-absorbent polymer gel and a substrate, but it is preferable to collect the biofilm after the entirety of the above-described region is covered with the biofilm in view of obtaining a large quantity of algal bodies of microorganisms. In addition, the collection may be performed after continuing culture for a while after the entirety of the region is covered with the biofilm

It is necessary to separate the water-absorbent polymer gel and the substrate from each other in order to collect microorganisms which have been cultured in the region between the water-absorbent polymer gel and the substrate. As the method for this, any well-known method can be used. For example, there is a method of separating a part of the substrate from the water-absorbent polymer gel by picking up the part of the substrate using forceps.

Microorganisms which have proliferated may be adhered to either or both of the water-absorbent polymer gel side and the substrate side. However, in general, the strength of the substrate is higher than that of the water-absorbent polymer gel, and therefore, it is preferable to make microorganisms adhere to the substrate side from the viewpoint of ease of detachment of microorganisms from the surface of the substrate.

[Detachment of Biofilm of Microorganisms from Surface of Water-Absorbent Polymer Gel or Substrate]

The detachment in the present invention is a type of a method of collection, and refers to treatment of peeling off microorganisms from the surface of a substrate or the surface of polymer gel.

As the method for detaching a biofilm of microorganisms from water-absorbent polymer gel or a substrate, any well-known method can be used, as long as the biofilm of microorganisms is detached from the surfaces thereof. Examples thereof include a method for peeling off a biofilm of microorganisms from the above-described surface using a cell scraper, a method of using a stream of water, and a method of using an ultrasonic wave in a liquid. The method of using a cell scraper is preferable. This is because, in other methods, the biofilm is diluted by a medium or the like, and therefore, there is a case where it is necessary to perform condensation again, which is inefficient.

[Collected Amount]

As the above-described collection method, greater than or equal to 70% of a biofilm of microorganisms is preferably collected, greater than or equal to 80% thereof is more preferably collected, greater than or equal to 90% thereof is still more preferably collected, and 100% thereof is most preferably collected. The collection rate of a biofilm of microorganisms can be confirmed by, for example, being visually recognized.

[Dry Microorganisms]

The dry microorganisms in the present invention are microorganisms obtained by drying a collected substance of microorganisms which have been obtained in accordance with the present invention. In the present invention, in a case where the microorganisms are microalgae, the microalgae are referred to as dry alga bodies.

As the method for drying the collected substance of microorganisms, any well-known method can be used as long as the method is a method through which it is possible to remove moisture in a collected substance of microorganisms, and there is no particular limitation. Examples thereof include a method of sun-drying a collected substance of microorganisms; a method of heat-drying a collected substance of microorganisms; a method of freeze-drying a collected substance of microorganisms; and a method of blowing dry air onto a collected substance of microorganisms. Among these, the freeze-drying method is preferable in view of being capable of suppressing decomposition of components contained in a collected substance of microalgae, and the heat-drying method or the sun-drying method is preferable in view of being capable of efficiently perform the drying in a short period of time.

[Moisture Content]

The moisture content of the present invention is obtained by, unless otherwise specified, dividing the weight (which can be generally calculated by subtracting the weight of a collected substance after drying (if necessary, the weight of a medium corresponding to a solid component is reduced) from the weight of the collected substance) of moisture contained in a collected substance by the weight of the collected substance, and then by multiplying 100.

The moisture content of a biofilm of microorganisms formed between water-absorbent polymer gel and a substrate in accordance with the present invention is preferably 10% to 95%, more preferably 30% to 90%, most preferably 50% to 70%. If the moisture content is 30% to 90%, it is easy to separate the biofilm from the substituent or the water-absorbent polymer gel, and the amount of energy required in a drying process is small.

[Useful Substance]

The useful substance in the present invention is one type of biomass derived from microorganisms and is the name of a substance beneficial to an industry which is obtained through a process such as an extraction process from biomass and a purification process. Such a substance includes a raw material, an intermediate, or a final product of a pharmaceutical product, cosmetics, or a health food, or the like; a raw material, an intermediate, or a final product of a chemical synthetic substance; a hydrocarbon compound; an energy-alternative substance such as oil, alcohol compound, hydrogen, or methane; oxygen; protein; nucleic acid; a lipid compound such as sugar or DHA; and astaxanthin. The useful substance can be accumulated in microalgae through a product accumulation process.

[Biomass]

The biomass in the present invention refers to a renewable organic resource derived from organisms excluding a fossil resource, and examples thereof include substances, food products, materials, fuel, and resources derived from organisms. As the algal biomass, residues of microalgae after the microalgae itself (which may also have a biofilm shape) and the useful substance have been collected are included.

[Oil]

Oil in the present invention refers to a flammable fluid substance, is a compound mainly formed of carbon and hydrogen, and is a substance occasionally containing oxygen atom, nitrogen atom, and the like. In general, oil is a mixed substance and is a substance which is extracted using a low-polarity solvent such as hexane, chloroform, or acetone. There is a case in which the composition thereof is formed of hydrocarbon compounds, fatty acids, triglycerides, or the like, and a case in which oil is formed of a plurality of types of compositions selected therefrom. In addition, oil can be esterified to be used as biodiesel.

The method of collecting a useful substance and oil contained in a collected substance of microorganisms is not particularly limited as long as the method does not impair the effect of the present invention.

As a general method for collecting oil, dry alga bodies are obtained by heat-drying a final collected substance, and then, oil is extracted using an organic solvent after performing cell-disruption. In general, the extracted oil contains impurities such as chlorophyll, and therefore, purification is performed. There is a case of performing purification through silica gel column chromatography or performing purification through distillation (for example, a distillation method disclosed in JP2010-539300A). In the present invention, it is possible to use such a method as well.

In addition, there is also a method of extracting oil in algal bodies using an organic solvent after crushing microorganisms through an ultrasonic treatment or dissolving microorganisms using protease or an enzyme (for example, a method disclosed in JP2010-530741A). In the present invention, it is possible to use such a method as well.

In addition, it is preferable that the microorganisms in the present invention have high oil content in view of usefulness as biomass. Specifically, the oil content per dry alga body of the microorganisms is preferably higher than or equal to 5 mass %, more preferably higher than or equal to 10 mass %, and particularly preferably higher than or equal to 15 mass %. In general, the oil content per the amount of dry microorganism of microorganisms is lower than or equal to 80 mass %.

### EXAMPLES

The present invention will be further described in detail with reference to the following Examples, but the present invention is not limited to the following Examples.

### Example 1

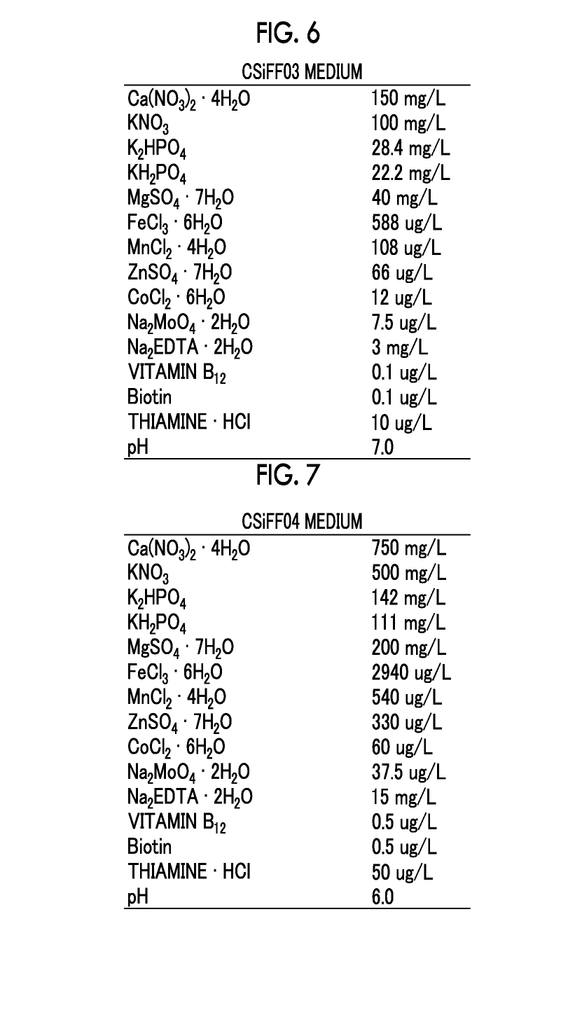

AVFF007 strains were cultured for 30 days under the amount of light of 4000 lux in a Purobio Petri dish (2-4727-01, As One Corporation) into which 65 mL of a CSiFF03 medium (FIG. 6) was put, a film-like structure on the liquid surface was collected through a deposition method using a nylon film, the collected film-like structure was put into a 5 mL tube for homogenizing ((TM-655S, Tomy Seiko Co., Ltd.) and was set in a beads cell disrupter (MS-100, Tomy Seiko Co., Ltd.: without using beads), and homogenization treatment lasting for 20 seconds was performed three times at 4200 rpm to obtain a suspension liquid a of AVFF007 strains.

This solution was diluted, the turbidity was calculated by measuring the absorbance at 660 nm, and the number of algal bodies of the above-described suspension liquid a was calculated from a relational expression between the turbidity and the number of algal bodies which have been calculated above. The number of algal bodies was 2.63×10⁸ cells/mL Therefore, 285 μL of the above-described suspension liquid a was collected in order to prepare a solution at 10×10⁴ cells/mL, and 750 mL of a suspension liquid b was obtained by mixing the collected suspension liquid a with a CSiFF03 medium.

16 Azunol Petri dishes (1-8549-04, As One Corporation) into which 45 mL of each suspension liquid b was put were prepared. Culturing was performed on the prepared Azunol Petri dishes under irradiation with fluorescent lamp at 4000 lux while repeatedly turning on and off the fluorescent lamp every 12 hours. Stationary culture was performed as the culturing and the culture temperature was 23° C.

6 days after the culturing, a form of a thin film-like substance was found on the surface of water. Therefore, the film-like structure on the surface of water was transferred by bringing a polyethylene film, which has been cut into the same size at the inner diameter of an Azunol Petri dish, come into contact with the film-like structure. The algal bodies on the polyethylene film were peeled off using a cell scraper, were placed on cover glass (2-176-13, As One Corporation) of which the weight was measured in advance, and were dried using a drier of which the temperature was set to be 100° C. After the drying, the weight of AVFF007 strains on the polyethylene film was measured after measuring the weight of the dried substance and subtracting the mass corresponding to the solid medium component contained in the medium therefrom. As a result of the measurement, the weight was 0.042 mg/cm². That is, the quantity of algal bodies to be used as seed algae became 0.042 mg/cm². An average value was used after performing two times of measurement.

Three sheets of the above-described polyethylene films to which each film-like structure formed of AVFF007 strains on the liquid surface was transferred were prepared. 1% agarose gel (as agarose, 15510-019, Invitrogen UltraPure Agarose™ was used) containing a CSiFF04 medium (FIG. 7) in an Azunol Petri dish was prepared. The agarose gel was obtained through gelation by performing autoclave treatment for 10 minutes at 121° C. after mixing the medium with the powder-like agarose, and by allowing the mixture to stand at room temperature. In addition, about 20 mL of a mixed solution of the agarose and the medium was used with respect to one Petri dish. In some cases, the agarose gel containing this medium is also called an agar medium.

A polyethylene film overlapped on agarose gel such that the surface of the agarose gel and the AVFF007 strain-adhered surface of the polyethylene film come into contact with each other; the Petri dish was put into a vacuum desiccator in a state of not being covered with an attached lid; and culturing was performed under irradiation with fluorescent lamp at 4000 lux while repeating light irradiation by turning on and off the fluorescent lamp every 12 hours at a carbon dioxide concentration of 5%. Stationary culturing was performed as the culturing, and a collecting process was performed at a culture temperature of 23° C., 14 days after the start of the culturing.

Microalgae (AVFF007 strains) proliferated in a region interposed between the agarose gel and the polyethylene film in accordance with the progress of the culturing, and a state was found that the microalgae had been colored green. After the completion of the culturing, the polyethylene film was peeled off from the top of the agarose gel. When visually observed, there was only a small quantity of microalgae on the agarose gel, and most of microalgae adhered to the top of the polyethylene film.

The microalgae on the surface of the polyethylene film were placed on cover glass of which the weight had been measured in advance while being peeled off from the polyethylene film using a cell scraper. The moisture content of microalgae became lower as it is possible to maintain the form thereof, and the microalgae were in a clayish solid state. The microalgae were dried using a drier of which the temperature was set to 100° C. After the drying, the weight of AVFF007 strains on the polyethylene film was measured after measuring the weight of the dried substance and subtracting the mass corresponding to the medium component contained in the medium therefrom. As a result of the measurement, the weight was 0.587 mg/cm². An average value was used after performing three times of measurement.

The moisture content of the collected substance in this Example was 50.8%. The moisture content of seed algae was 92.9%, and the moisture content of a collected substance using water surface-floating culture method in Comparative Example 1 was 81.7%.

The moisture content of a collected substance in a case where microalgae are cultured through floating culture method using a centrifugal separator is about 90%. Therefore, it is considered that the moisture content in this Example is extremely low, and therefore, the efficiency in an oil extraction process becomes extremely high. The amount of water in the moisture content of 50.8% in the present invention becomes one ninth of the amount of moisture in the moisture content of 90% in the method using a centrifugal separator of the conventional method, and therefore, the effect of the amount of moisture reduced in the present invention is considerable.

### Comparative Example 1

### Liquid Surface-Floating Culture

This Comparative Example relates to the quantity of algal bodies on the liquid surface in a case where liquid surface-floating culture has been performed, and the moisture content after the algal bodies have been collected.

Culturing was performed through the same method as that in Example 1 immediately before collecting seed algae. The number of samples is 2.

In Example 1, the film-like structure on the surface of water was transferred using a polyethylene film. However, in this Example, culturing was continued as it was without transferring the film-like structure. That is, the water surface-floating culture was continued as it was. The culturing was performed under the same conditions, such as culture conditions and the quantitative determination of a collected substance, as those in Example 1.

The yield became 0.678 mg/cm². The moisture content became 82%. Although the yield was slightly higher than that of Example 1, the moisture content was significantly increased.

### Comparative Example 2

### Liquid Surface-floating Culture+Coating of Substrate

This Example is a Comparative Example in a case of using a liquid medium instead of agarose gel.

Films to which suspension liquids a and b, and seed algae a were prepared through the same method as that in Example 1.

One silicone rubber sheet to which a film-like structure formed of AVFF007 strains on the liquid surface was transferred, overlapped on 1% agarose gel containing a CSiFF04 medium which had been made in an Azunol Petri dish, and the other silicone rubber sheet to which a film-like structure formed of AVFF007 strains on the liquid surface was transferred, overlapped on a container into which 45 mL of a CSiFF04 medium was put into an Azunol Petri dish, such that the surface of the agarose gel or the medium and the AVFF007 strain-adhered surface on the silicone rubber sheet come into contact with each other; the Petri dish was put into a vacuum desiccator in a state of not being covered with an attached lid; and culturing was performed under irradiation with fluorescent lamp at 4000 lux while repeatedly turning on and off the fluorescent lamp every 12 hours at a carbon dioxide concentration of 5%. Stationary culture was performed as the culturing, and a collecting process was performed at a culture temperature of 23° C., 14 days after the start of the culturing. The silicone rubber sheet, with which the liquid medium was coated, floated on the liquid surface during the culturing.

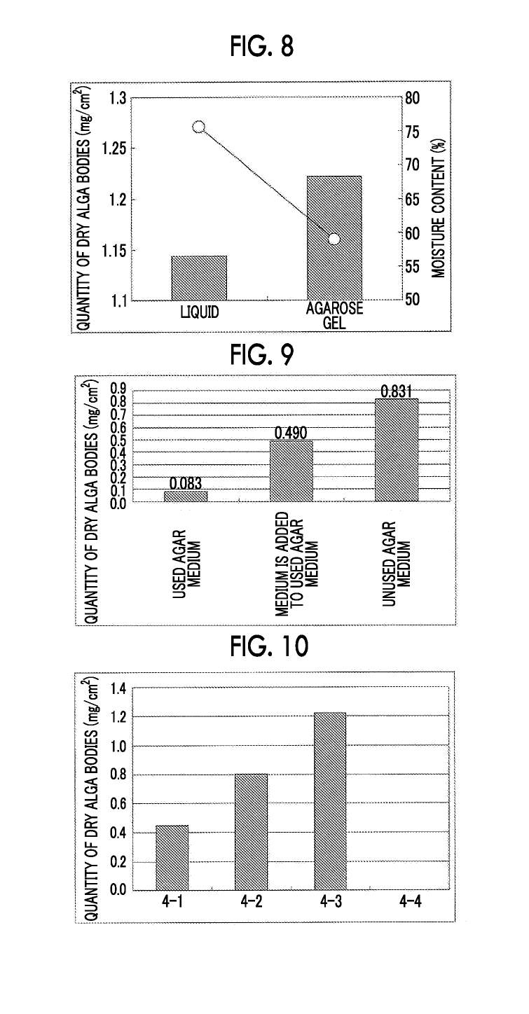

The results are shown in FIG. 8. In a case where a liquid medium was coated with a substrate and in a case where agarose gel in the present invention was coated with a substrate, the quantity of algal bodies of the latter after proliferation was increased. In addition, the moisture content of the case in which the method of the present invention was used was decreased. Therefore, it is considered that the amount of energy input in the drying process can be significantly reduced. The bar graph represents the quantity of dry alga bodies and the symbol O represents the moisture content of the collected substance.

### Example 2

### Reuse of Agar Medium

This Example is about a case in which agarose gel which has been used once is used and it is verified whether or not it is possible to reuse agarose gel by adding a small amount of nutrients to the agarose gel which has been used once and performing second primary culture.

A suspension liquid a (corresponding to the suspension liquid a in Example 1) was obtained through the same method as that in Example 1. In addition, a suspension liquid b (corresponding to the suspension liquid b in Example 1) was obtained through the same method as that in Example 1. However, the number of algal bodies was set to 5.91×10⁸ cells/mL. Therefore, 186 μL of the above-described suspension liquid a was collected in order to prepare a solution at 10×10⁴ cells/mL, and 1100 mL of the suspension liquid b was obtained.

Water surface-floating culture was performed through the same method as that in Example 1 using Azunol Petri dishes. However, 18 Petri dishes were prepared.

A film to which seed algae a were adhered was prepared through the same method as that in Example 1. However, a silicone rubber sheet was used as the type of film for a culture period of 3 days, and the quantity of seed algae became 0.008 mg/cm².

A first primary culturing process was performed through the same method as that in Example 1 using an agar medium. However, a collecting process was performed 14 days after the culturing. The collected amount was 1.222 mg/cm² and the moisture content was 59.0%. The agar medium after the collection of algal bodies was secured in order to use a part of the agar medium in second primary culture.

Hereinafter, pre-culture for performing the second primary culture was performed.

A suspension liquid c (corresponding to the suspension liquid a in Example 1) was obtained through the same method as that in Example 1.

A suspension liquid d (corresponding to the suspension liquid b in Example 1) was obtained through the same method as that in Example 1. However, the number of algal bodies was set to 2.73×10⁸ cells/mL. Therefore, 251 μL of the above-described suspension liquid c was collected in order to prepare a solution at 10×10⁴ cells/mL, and 685 mL of the suspension liquid d was obtained.

Liquid surface-floating culture was performed through the same method as that in Example 1 using Azunol Petri dishes. However, 12 Petri dishes were prepared.

Hereinafter, the second primary culture process was performed.

A film to which seed algae b were adhered was prepared through the same method as that in Example 1. However, a silicone rubber sheet was used as the type of film for a culture period of 3 days, and the quantity of algal bodies used as seed algae became 0.006 mg/cm².

Culturing corresponding to the second primary culture process was performed through the same method as that in Example 1 using unused agarose gel. However, a silicone rubber sheet was used as the type of film, AVFF007 strains derived from seed algae b were used, and a collecting process was performed 14 days after the culturing.

The second primary culture process was performed through the same method as that in Example 1 using agarose gel after the collecting process which was used in the first primary culture. However, a silicone rubber sheet was used as the type of film, AVFF007 strains derived from seed algae b were used, and a collecting process was performed 14 days after the culturing.

The second primary culture process was performed through the same method as that in Example 1 using agarose gel after the collecting process which was used in the first primary culture. However, 2 mL of a CSiFF04 medium was added to the agarose gel per Petri dish. A silicone rubber sheet was used as the type of film, AVFF007 strains derived from seed algae b were used, and a collecting process was performed 14 days after the culturing.

The results were shown in FIG. 9. The quantity of algal bodies collected in a case of using used agarose gel was 0.083 mg/cm², whereas the quantity of algal bodies collected in a case of using unused agarose gel was 0.831 mg/cm². The collected amount became about one tenth. It is considered that this is because nutrient components required in the second primary culture process were insufficient, since the nutrient components in agarose gel were consumed in the first primary culture process. In contrast, in a sample which was obtained by adding a medium to agarose gel, the collected amount was about 60% compared to the case of using an unused agar medium, the collected amount became about 6 times with respect to a case in which no medium was added to the used agar medium.

From the above, it was found that it was possible to reuse used agarose gel by adding a medium thereto.

### Example 3

### Reuse of Substrate

This Example is Example for verifying whether or not it is possible to reuse a substrate, or whether or not it is possible to use algal bodies remaining on the substrate in a small quantity at the time of reuse.

A suspension liquid a was obtained through the same method as that in Example 1. A suspension liquid b was obtained through the same method as that in Example 1. However, the number of algal bodies was set to 1.16×10⁸ cells/mL. Therefore, 268 μL of the above-described suspension liquid a was collected in order to prepare a solution at 10×10⁴ cells/mL, and 3100 mL of the suspension liquid b was obtained.

Water surface-floating culture was performed through the same method as that in Example 1 using Azunol Petri dishes. However, 36 Petri dishes were prepared.

A film to which seed algae a were adhered was prepared through the same method as that in Example 1. However, a silicone rubber sheet was used as the type of film for a culture period of 5 days, and the quantity of seed algae became 0.003 mg/cm².

A first primary culturing process was performed through the same method as that in Example 1. However, a collecting process was performed 14 days after the culturing. The collected amount was 2.15 mg/cm² and the moisture content was 71.0%.

The algal body-adhered surface of the silicone rubber sheet, to which a slight quantity of microalgae was considered to be adhered after the above-described collection, was pasted to new agarose gel so as to be brought into contact therewith. That is, it was verified whether it is possible to perform culturing using a very small quantity of algal bodies (that is, considering the algal bodies as input algal bodies) remaining on a substrate after coating the surface of the new agarose gel with the substrate on which algal bodies were removed. In this state, a second primary culture process was performed through the same method as that of the primary culturing process in Example 1.

The quantity of algal bodies 14 days after the culturing was measured, and the results were that the collected amount was 1.971 mg/cm² and the moisture content was 72.1%.

From the above, it became clear that it is possible to perform culturing again using microalgae remaining on a substrate after collection without newly supplying seed algae to the substrate.

### Example 4

### Coating+Substrate Coating

This Example is Example in a case where culturing is performed by directly coating water-absorbent polymer gel with microalgae without using a substrate, in a case where culturing is performed by directly coating water-absorbent polymer gel with microalgae using a substrate, in a case where culturing is performed through the method of the present invention, or in a case where culturing is performed without using water-absorbent polymer gel.

A suspension liquid a was obtained through the same method as that in Example 1. A suspension liquid b was obtained through the same method as that in Example 1. However, the number of algal bodies was set to 5.91×10⁸ cells/mL. Therefore, 186 μL of the above-described suspension liquid a was collected in order to prepare a solution at 10×10⁴ cells/mL, and 1100 mL of the suspension liquid b was obtained.

Water surface-floating culture was performed through the same method as that in Example 1 using Azunol Petri dishes. However, 18 Petri dishes were prepared.

A film to which seed algae a were adhered was prepared through the same method as that in Example 1. However, a silicone rubber sheet was used as the type of film for a culture period of 3 days, and the quantity of seed algae became 0.008 mg/cm². That is, the quantity of algal bodies which was input and used in the culturing was 0.008 mg/cm².

Algal bodies were peeled off from a silicone rubber sheet, to which seed algae a were adhered, using a cell scraper, and the surface of the agarose gel was directly coated with the peeled off algal bodies. That is, this is an example in a case where culturing was performed by coating the top of an agar medium with the same quantity of algal bodies as that of the algal bodies adhered to the substrate without using the substrate. This sample was set to a sample 4-1.

A sample, which was prepared such that algal bodies were peeled off from a silicone rubber sheet, to which seed algae a were adhered, using a cell scraper, the surface of the agarose gel was directly coated with the peeled off algal bodies, and the surface coated with the algal bodies were coated with a silicone rubber sheet which had been cut into a size of the inner diameter of the Azunol Petri dish, was set to a sample 4-2.

A sample, in which the surface of the agarose gel was coated with a silicone rubber sheet, to which seed algae a were adhered, such that the algal bodies and the agarose gel come into direct contact with each other, was set to a sample 4-3.

A silicone rubber sheet, to which seed algae a were adhered, was made to overlap on the surface of Azunol Petri dish such that the AVFF007 strain-adhered surface on the silicone rubber sheet and the surface of the Azunol Petri dish come into direct contact with each other. That is, this is an example of culturing in a case where there is no agarose gel. This sample was set to a sample 4-4.

The above-described samples 4-1 to 4-4 were cultured through the same method as that in Example 1.

The culturing and collecting were performed through the same method as that in Example 1.

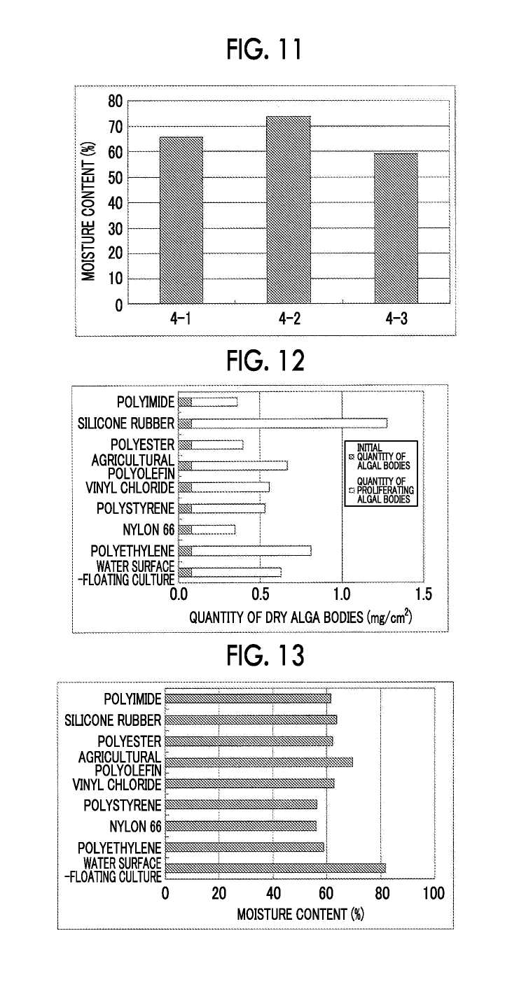

The results of the quantity of algal bodies were shown in FIG. 10 and the moisture content at this time was shown in FIG. 11. In the case of the sample 4-1, the quantity of algal bodies was 0.45 mg/cm², which shows that the amount of proliferation was larger than that of the sample 4-4, but was smaller than that of the sample 4-2. It is estimated that this is because the form of algal bodies on the agarose gel is in a colony shape, and due to this shape, it is impossible to effectively use the surface area, and therefore, the quantity of algal bodies was not increased. It was considered that, in the case of sample 4-2, the coating of the agarose gel with algal bodies was not uniform compared to the sample 4-3, and therefore, it was impossible to effectively use the surface area, and the quantity of algal bodies was limited from being increased. That is, this shows that it is preferable to use a microalga-adhered substrate which was prepared by transferring a biofilm of microalgae on the liquid surface cultured through liquid surface-floating culture, to the top of the surface. In contrast, in the case of the sample 4-3, it was possible to obtain the largest quantity of algal bodies. It is considered that this is because it was possible to effectively use the surface of the agarose gel since the form of algal bodies after proliferation was in a film shape. In the case of the sample 4-4, microalgae did not proliferate at all. It is estimated that this is because the moisture and the nutrient source were insufficient since there was no water-absorbent polymer gel.

### Example 5

### In Case of Using Various Films

In this Example, an influence of use of various films as substrates on proliferating properties.

A suspension liquid a was obtained through the same method as that in Example 1. A suspension liquid b was obtained through the same method as that in Example 1. However, the number of algal bodies was set to 1.51×10⁸ cells/mL Therefore, 610 μL of the above-described suspension liquid a was collected in order to prepare a solution at 10×10⁴ cells/mL, and 920 mL of the suspension liquid b was obtained.

Liquid surface-floating culture was performed through the same method as that in Example 1 using Azunol Petri dishes. However, 20 Petri dishes were prepared.

A film to which seed algae were adhered was prepared through the same method as that in Example 1. However, a silicone rubber sheet was used as the type of film for a culture period of 3 days, and the quantity of seed algae became 0.078 mg/cm². The quantity of algal bodies input was 0.078 mg/cm².

A primary culturing process was performed through the same method as that in Example 1. However, as the type of film, a film shown in FIG. 12 was used, and a collecting process was performed 18 days after the start of the primary culturing process.

A state, in which microalgae proliferate in a region interposed between the agarose gel and various films in accordance with the progress of the culturing, was found. In a case of a polyethylene terephthalate film, the color of microalgae was yellowish green, in the case of the silicone rubber sheet, the color of microalgae was yellowish red, and in cases of other films, the color of microalgae was an intermediate color between the both colors thereof. After the completion of the culturing, the various films were peeled off from the top of the agarose gel. When visually observed, there were almost no microalgae on the agarose gel, and most of microalgae adhered to the top of the film.

The quantitative determination was performed through the same method as that in Example 1. The results were shown in FIG. 12. The silicone rubber sheet had the highest amount of proliferation. It is considered that this is because the carbon dioxide permeability of the silicone rubber sheet was high compared to other films.

The results of the moisture content of collected substances were shown in FIG. 13, and were between 55% and 70%. The moisture content was extremely low compared to the method using a conventional centrifugal separator. Therefore, it is possible to very simply perform collection since the collected substance is adhered to the film in a film shape, and thus, this is useful for reducing costs in an oil extraction process.

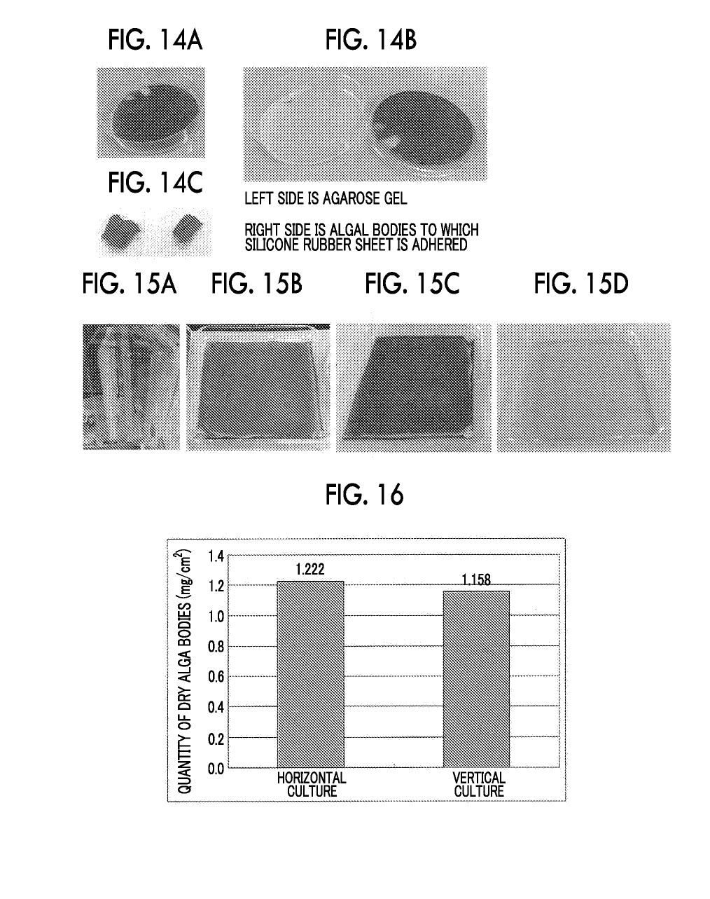

A state of a collected substance immediately before the collection in the case of using the silicone rubber sheet was shown in FIG. 14A, a state of the collected substance after peeling of the silicone rubber sheet from the agarose gel was shown in FIG. 14B, and a state of the collected substance after detaching microalgae from the silicone rubber sheet was shown in FIG. 14C. Most microalgae are adhered on the silicone rubber sheet side as shown in FIG. 14B. As shown in FIG. 14C, the amount of moisture is low to the extent that it is possible to maintain the form of the collected substance while collecting the aggregation of microalgae as shown in FIG. 14C since the moisture content is low.

### Example 6

This Example, is Example in which drying is performed using a fact that the surface area of microalgae is wide in order to aim further reduction in moisture content of microalgae on a substrate, and microalgae detached from the substrate.

A sample prepared by having a silicone rubber sheet of Example 5 as a substrate, that is a sample in which a biofilm of microalgae was adhered to the top of a substrate as shown in FIG. 14B, and a sample in which the biofilm of microalgae was detached from the top of the substrate as shown in FIG. 14C were placed on a balance by adjusting the amount of light of an artificial sunlight (Probright V, NIPPON PAINT Co., Ltd.) to 15000 lux. Then, the moisture content was calculated after measuring the weight of the samples for every constant time. The moisture content was calculated after calculating the dry weight after the samples were completely dried using a drier. In addition, the room temperature was 24.1° C. and the humidity was 46%.

The moisture content of microalgae on the substrate was 58% at the time of starting irradiation, 36% after 5 minutes, 21% after 10 minutes, and 16% after 20 minutes. In contrast, the moisture content of microalgae detached from the substrate was 60% at the time of starting irradiation, 51% after 5 minutes, 42% after 10 minutes, and 36% after 20 minutes. From the above, it was possible to further reduce the moisture content of all of the samples. In addition, it was possible to significantly reduce the moisture content of microalgae on the substrate in which it was considered that the surface area was larger than that of microalgae detached from the substrate.

### Example 7

In this Example, culturing is performed by coating one continuous surface of water-absorbent polymer gel with at least two or more substrates.

Suspension liquids a and b were obtained and liquid surface-floating culture was performed through the same method as that in Example 1 using an Azunol Petri dish. A silicone rubber sheet which was used as the type of film and to which seed algae a were adhered was prepared by making microalgae on the liquid surface adhere to the silicone rubber sheet using a transferring method. The quantity of algal bodies thereof was measured and was 0.012 mg/cm². The quantity of algal bodies which was input and used in the culturing was 0.012 mg/cm².

Culturing was performed by pasting the silicone rubber sheet, to which the seed algae a were adhered, on the agarose gel under the same culture conditions as those in Example 1.

Furthermore, silicone rubber sheets which were divided into four sheets and to which seed algae a were adhered were prepared. The quantity of algal bodies was one fourth of the above-described quantity of algal bodies. Similarly, culturing was performed by pasting the silicone rubber sheets to agarose gel under the same culture conditions as those in Example 1. That is, the four silicone rubber sheets were pasted on one sheet of water-absorbent polymer gel such that gaps of about 0.5 mm are generated between the silicone rubber sheets.

The quantity of algal bodies in the case where one silicone rubber sheet was pasted on the water-absorbent polymer gel became 1.3 mg/cm², but the quantity of algal bodies in the case where four silicone rubber sheets were pasted thereon became 1.6 mg/cm². It is estimated that this is because, in the latter case, carbon dioxide is supplied through the gaps between the films, and gas generated in accordance with the culturing promptly flows to the outside of a culture vessel.

### Example 8

In this Example, microorganisms are cultured between water-absorbent polymer gel and a substrate having an uneven structure using the structure having the above-described uneven structure.

Suspension liquids a and b were obtained and liquid surface-floating culture was performed through the same method as that in Example 1 using an Azunol Petri dish. A polyethylene film, to which seed algae a were adhered and which has an uneven structure, and a polyethylene film, to which seed algae a were adhered and which does not have an uneven structure, were prepared by making microalgae on the liquid surface adhere on the polyethylene films through a transferring method using the polyethylene film having the uneven structure, as the type of film. The quantity of algal bodies was measured, and the result was 0.012 mg/cm². That is, the quantity of algal bodies which was input and used in the culturing was 0.012 mg/cm². The polyethylene film having unevenness was prepared by being scrubbed using commercially available sandpaper.

Culturing was performed under the same culture conditions as those in Example 1 after pasting the polyethylene films, to which seed algae a were adhered, on agarose gel.

The quantity of dry alga bodies in the case where the polyethylene film having no uneven structure was pasted on the agarose gel was 0.65 mg/cm², but the quantity of dry alga bodies in the case where the polyethylene film having an uneven structure was pasted on the agarose gel was 0.82 mg/cm². It is estimated that this is because, in the latter case, carbon dioxide is supplied through the gaps between the film and a biofilm of microalgae, and gas generated in accordance with the culturing promptly flows to the outside of a culture vessel.

### Example 9

### In Case of Haematococcus (Other Microalgae)

In this Example, the method of the present invention was applied to Haematococcus for which it is impossible to perform liquid surface-floating culture. That is, it is shown that, in the method of the present invention, it is possible to use various types of microalgae.

A part of NIES-2264 (Haematococcus lacustris) which had been cultured in a 100 mL conical flask was collected, the number of algal bodies was measured using a hemocytometer after being diluted using the same medium, and the concentration was prepared. Then, the top of agarose gel containing a CSiFF04 medium was coated with the part of NIES-2264 so as to have a concentration of 1×10⁴ cells/cm². The coating was performed so as to be as even as possible using a disposable stick (1-4633-12, As One Corporation) after adding dropwise an alga body solution using a pipette.

After the coating with the alga body solution, the alga body solution was coated with a silicone rubber sheet which was cut into a circular shape so as to have the same area as that of the inner wall of an Azunol Petri dish, and bubbles formed between the sheet and the agar medium were removed as much as possible to start culturing.

The culturing was performed such that a plastic Petri dish coated with microalgae was put into a vacuum desiccator, and a lid of the vacuum desiccator was closed after installing an opening portion of the plastic Petri dish toward an upper side, that is, a light source side in a state in which a lid attached to the plastic Petri dish was removed, and after setting the concentration of carbon dioxide to 5%. The culturing was performed under the same conditions as those in Example 1 as other culture conditions. The top of the agarose gel within the Petri dish became a green color in accordance with the culturing, and the algal bodies were collected from the agarose gel 14 days after the culturing. The silicone rubber sheet was peeled off from the top of the agarose gel, and most algal bodies were adhered on the silicone rubber sheet. Therefore, the microalgae were collected using a cell scraper from the top of the silicone rubber sheet. The moisture content of the collected substance after freeze-drying was calculated and was 78.4%. In addition, the quantity of dry alga bodies was 3.7 mg/cm².

The obtained dry alga bodies were put into a 2 mL tube for homogenizing (TM-626, Tomy Seiko Co., Ltd.), 0.6 g of glass beads with a diameter of 0.5 mmφ and 1 mL of hexane were put thereinto, and the tube was covered with a lid. Then, the tube was set in a beads cell disrupter (MS-100, Tomy Seiko Co., Ltd). After performing homogenization treatment, lasting for 20 seconds, three times at 5500 rpm, a container was centrifugally removed. Then, a supernatant was put into a 2 mL glass sample bottle, and centrifugal treatment was performed again. Again, a supernatant was put into a 2 mL glass sample bottle of which the weight had been previously measured, and the solvent was removed. Then, the amount of the remaining viscous substance was regarded as the amount of oil. The amount of oil was 12.2% with respect to the quantity of dry alga bodies.

From the above, it was determined that it was also possible to culture algal bodies other than AVFF007 strains through a primary culture method, and it was also possible to perform culturing, even through a culture method other than liquid surface-floating culture.

NIES-2264 does not form a film-like structure on the liquid surface.

### Example 10

A suspension liquid a and 1100 mL of a suspension liquid b were obtained by performing pre-culture through the same method as that in Example 1.

Six Petri dishes into which 65 mL of a suspension liquid b was put were prepared, and culturing was performed through the same method as that in Example 1. However, Purobio Petri dishes were used instead of Azunol Petri dishes.

A biofilm of microalgae on the liquid surface was transferred to a silicone rubber sheet through the same method as that in Example 1, and the dry weight thereof was measured. As a result of the measurement, the dry weight became 0.0075 mg/cm². That is, the quantity of algal bodies used as seed algae became 0.0075 mg/cm².

Agarose gel was prepared and culturing was performed through the same method as that in Example 1. However, two Purobio Petri dishes out of four Purobio Petri dishes were installed horizontally with respect to the ground, and the remaining two Purobio Petri dishes were installed vertically with respect to the ground. The installation interval between the Purobio Petri dishes in the case where the Purobio Petri dishes were vertically installed was set to 1.5 cm.

Microalgae proliferated in a region interposed between the agarose gel and the silicone rubber sheet in accordance with the progress of the culturing, and a state was found that the microalgae had been colored in green. After the completion of the culturing performed for 14 days, the silicone rubber sheet was peeled off from the top of the agarose gel. When visually observed, there was only a small quantity of microalgae on the agarose gel, and most of microalgae adhered to the top of the silicone rubber sheet. A state during culturing 7 days after the start of the culturing was shown in FIG. 15A, an agarose gel-AVFF007 strain-silicone rubber sheet structure 7 days after the start of the culturing was shown in FIG. 15B, an AVFF007 strain-silicone rubber sheet structure after being peeled off from the agarose gel 7 days after the start of the culturing was shown in FIG. 15C, and the agarose gel after collection was shown in FIG. 15D. In FIG. 15A, four substrates are installed, and are substrates in which microalgae are not adhered to both ends of each substrate.

The dry weight thereof was measured through the same method as that in Example 1.

The results were shown in FIG. 16. In a case where the Purobio Petri dishes were installed horizontally with respect to the ground, the quantity of algal bodies was slightly larger compared to the case where the Purobio Petri dishes were vertically installed. It was considered that this was because the amount of light which the algal biomass-adhered surface received was large in the former case.

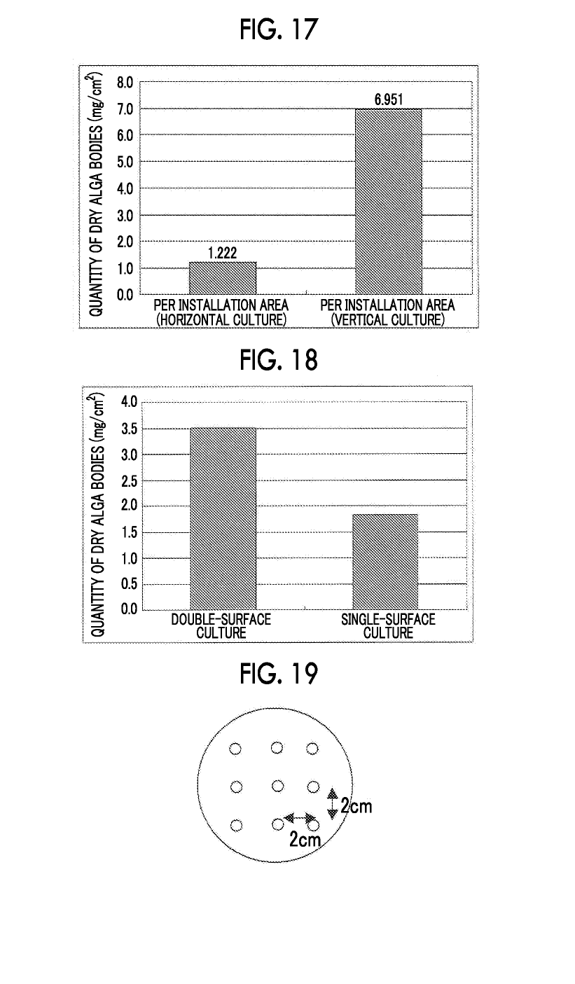

The results which are obtained after the results in FIG. 16 are converted into quantity of algal bodies per installation area are shown in FIG. 17. In the case of the vertical culture, the Purobio Petri dishes were installed at an interval of 1.5 cm, and therefore, the quantity of algal bodies per installation area were increased and became 5.7 times of the quantity of algal bodies compared to the case where the Purobio Petri dishes were horizontally installed. The results shows that it is possible to more effectively use the culture area using the method of the present invention. The moisture content became 59.0% in the case of the horizontal installation, and 62.9% in the case of the vertical installation. The moisture content was more significantly decreased than the moisture content which was about 90% and was generally obtained through collection using a centrifugal separator or the like.

The oil content became 22.1% in terms of weight proportion per dry alga body.

### Example 11

### Adhesion on Both Surfaces

Pre-culture, preparation of an alga body suspension liquid, and preparation of a biofilm were performed through the same method as that in Example 7. The amount of the biofilm was 0.003 mg/cm². That is, the quantity of algal bodies used as seed algae became 0.003 mg/cm².

Similarly to Example 7, a biofilm of microalgae of AVFF007 strains was transferred to a single surface of a silicone rubber sheet using the silicone rubber sheet as a substrate.

An agarose gel layer containing CSiFF04 media on both surfaces of a polystyrene plate was prepared. That is, the polystyrene plate was a support substrate, and the agarose gel layer was water-absorbent polymer gel. The AVFF007 strain-adhered silicone rubber sheet was pasted on the surface of this agarose gel. That is, a structure consisting of a silicone rubber sheet, an alga body layer, agarose gel, a polystyrene plate, agarose gel, an alga body layer, and a silicone rubber sheet was formed. Four sheets of this structure were installed such that the alga body layers of the structure became vertical with respect to the ground, and were put into a vacuum desiccator. After adjusting the concentration of carbon dioxide to 5%, the structure was irradiated with a fluorescent lamp at the amount of light of 15000 lux. The irradiation with light was set to be repeatedly turned on and off at a time interval of 12 hours.

Similarly, a structure in which microalgae were adhered to a single surface was also prepared.

As a result of culturing, the quantity of algal bodies per culture area became 1.832 mg/cm² in single-surface culture and became 3.504 mg/cm² in double-surface culture. In this manner the efficiency of the double-surface culture was improved than that of the single-surface culture. The results for each quantity of dry alga bodies were shown in FIG. 18. In addition, in the result for each installation area, the former became 10.44 mg/cm² and the latter became 20.00 mg/cm².

### Example 12

### In Case Where Film is Bored with Hole

Similarly to Example 7, a suspension liquid a and a suspension liquid b were prepared by performing pre-culture.

Four Petri dishes into which 45 mL of each suspension liquid b was put were prepared, and culturing was performed through the same method as that in Example 7. However, Azunol Petri dishes were used instead of Purobio Petri dishes.

A biofilm of microalgae on the liquid surface was transferred to a silicone rubber sheet through the same method as that in Example 7, and the dry weight thereof was measured. As a result of the measurement, the dry weight became 0.0075 mg/cm². That is, the quantity of algal bodies used as seed algae became 0.0075 mg/cm².

Agarose gel was prepared and culturing was performed through the same method as that in Example 7. However, four sheets of polyethylene films were prepared, and films for which no treatment was done were used as two sheets out of the four sheets of the polyethylene films and films bored with 9 holes in total using a needle were used as the remaining two sheets (FIG. 19). The interval of the holes was set such that the central portions of the films were bored with 3×3 holes at an interval of 2 cm. Culturing was performed on these films through the same method as that in Example 1.

Microalgae proliferated in a region interposed between the agarose gel and the polyethylene film in accordance with the progress of the culturing, and a state was found that the microalgae had been colored in green. After the completion of the culturing performed for 14 days, the polyethylene film was peeled off from the top of the agarose gel.

The quantitative determination was performed through the same method as that in Example 1.

The results were shown in FIG. 20. The quantity of algal bodies became 0.48 mg/cm² in the case where there was no hole, and became 0.61 mg/cm² in the case where there were holes. From these results, it is considered that the existence of holes in a film for coating has an action for increasing the amount of proliferation. It is considered that this is because supply of carbon dioxide required for proliferation is limited in the case where there is no hole in a film for coating, but it is possible to take carbon dioxide through holes and to release oxygen generated in accordance with proliferation to the outside of the culture system in the case of a film provide with holes at an adequate interval.

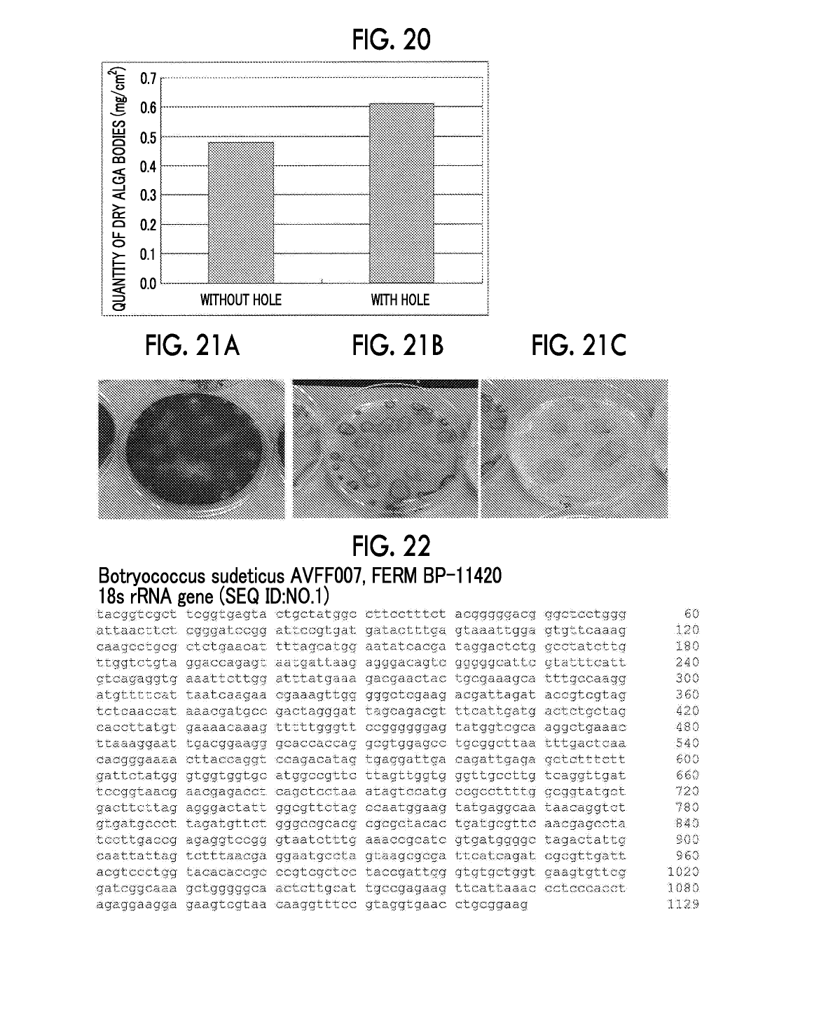

This is because many gas phases of the oxygen, which was generated in accordance with proliferation, are generated in a region between agarose gel and a film as shown in FIGS. 21A to 21C, and in a case where there is no hole, it becomes difficult for algal bodies to be separated from the film in accordance with the progress of drying of the algal bodies (FIG. 21A). In addition, it is easy for the algal bodies to remain, even on the agarose gel after the separation of the film (FIG. 21B, and therefore, the quantity of algal bodies collected decreases. In contrast, it was considered that, in the case of using a film bored with holes, even if a small number of bubbles or a small number of algal bodies on the agarose gel remaining can be found, the extent thereof is small, and as a result, the quantity of algal bodies collected is improved compared to the former case (FIG. 21C). In this manner, the quantity of algal bodies collected is improved by boring holes on a film. In a case of using a film, such as a silicone rubber sheet with high gas permeability, such a problem is rarely caused.

### Example 13

### In Cases of FFG039 Strains and Diatoms

Culturing was performed through the same method as that in Example 9. However, three kinds of microalgae in total, for example, Chlorococcum sp., FFG039 strains, NIES-2199 (Botryococcus braunii) used as green algae, and NIES-1339 (Nitzschia sp.) used as diatoms, were cultured.

In addition, a CSiFF04 medium was used in the case of FFG039 strains, a C medium was used in the case of NIES-2199, and an f/2 medium was used in the case of NIES-1339.

After culturing, the moisture contents of collected substances of biomass adhered to films were mainly 63.2%, 65.1%, and 61.9%, and the quantities of dry alga bodies were 5.2 mg/cm², 2.7 mg/cm², and 3.6 mg/cm².

From the above, it was found that it was possible to perform culturing, even with the FFG039 strains, Botryococcus sp., and the diatoms.

### Example 14

### In Case of Microorganisms

Culturing was performed through the same method as that in Example 9. However, yeast (101399, Candida utilis, Wako Pure Chemical Industries) was used as microorganisms. As the culture method, a culture method of Microbiol. Cult. Coll. 25(2): 89-91, 2009 was followed. An agar medium was prepared as a YM liquid medium, and culturing was performed for 5 days at a temperature of 30° C. In addition, the medium was not intentionally irradiated with light, and shaking was not performed. After the culturing, the collected amount of biomass adhered to a film was mainly 4.7 mg/cm².

### Sequence Table Free Text

SEQ ID No: 1: Part of base sequence of 18S rRNA gene of AVFF007 strains

SEQ ID No: 2: Part of base sequence of 18S rRNA gene of FFG039 strains

1. A method for culturing microorganisms comprising:
a step of seeding microorganisms on at least a part of the surface of the water-absorbent polymer gel;
a step of coating a region on the water-absorbent polymer gel on which at least microorganisms are seeded, with the substrate; and
a step of culturing the seeded microorganisms between the substrate and the surface of the water-absorbent polymer gel.

2. The culture method according to claim 1,
wherein microorganisms form a biofilm through culturing.

3. The culture method according to claim 1,
wherein the microorganisms are microalgae which can be subjected to liquid surface-floating culture, and the seeding of microalgae on the surface of the water-absorbent polymer gel is performed by coating the at least the part of the surface of the water-absorbent polymer gel with a substrate to which a biofilm formed on the liquid surface through liquid surface-floating culture is transferred, or by transferring the biofilm formed on the liquid surface through liquid surface-floating culture to the surface of the water-absorbent polymer gel.

4. The culture method according to claim 1,
wherein seeding of microorganisms on the surface of the water-absorbent polymer gel is performed by coating the at least the part of the surface of the water-absorbent polymer gel with a substrate which has been immersed in a microorganism suspension liquid, by immersing the surface of the water-absorbent polymer gel in the microorganism suspension liquid, or by spraying or coating at least either of the surface of the water-absorbent polymer gel or the substrate with microorganisms.

5. The culture method according to claim 1,
wherein the culturing is performed using both surfaces of the water-absorbent polymer gel.

6. The culture method according to claim 1, further comprising:
a step of collecting microorganisms after the culturing.

7. The culture method according to claim 6, further comprising:
a step of re-using the water-absorbent polymer gel or the substrate in culturing after the collection of microorganisms.

8. The culture method according to claim 6, further comprising:
a culture step in which microorganisms remaining on the water-absorbent polymer gel or the substrate after the collection of microorganisms, are used as seed microorganisms.

9. The culture method according to claim 6, further comprising:
a step of reducing the moisture content while maintaining an obtained collected substance to be adhered to the substrate or after making the collected substance be detached from the substrate,
wherein the collection of the microorganisms after the culturing is performed by removing the substrate, to which the microorganisms are adhered, from the water-absorbent polymer gel.

10. The culture method of microorganisms according to claim 1,
wherein the carbon dioxide permeability of the substrate is greater than or equal to 500 cc/m²·24 h/atm.

11. The culture method according to claim 1,
wherein the culture method is vertical culture, in which the surface of the water-absorbent polymer gel is maintained in a vertical direction, or horizontal culture in which the surface of the water-absorbent polymer gel is maintained in a horizontal direction.

12. The culture method according claim 1,
wherein holes are bored at least at one or more sites on the substrate.

13. The culture method according to claim 1,
wherein an uneven structure is formed in at least a part of a region on at least one of the substrate and the water-absorbent polymer gel.

14. The culture method according to claim 1,
wherein at least a part of the surface of the water-absorbent polymer gel is coated with a plurality of substrates.

15. The culture method according to claim 1,
wherein the microorganisms are eumycetes, green algae, or diatoms.

16. The culture method according to claim 1,
wherein the microorganisms belong to yeast, Botryococcus sp., Chlamydomonas sp., Chlorococcum sp., Chlamydomonad sp., Tetracystis sp., Characium sp., Protosiphon sp., or Haematococcus sp.

17. The culture method according to claim 1,
wherein the microorganisms belong to the same species as that of Botryococcus sudeticus or Chlorococcum sp. FERM BP-22262.

18. The culture method according to claim 1,
wherein the microorganisms are Botryococcus sudeticus FERM BP-11420 or microalgae strains having taxonomically the same properties as those of Botryococcus sudeticus FERM BP-11420, or are Chlorococcum sp. FERM BP-22262 or microalgae strains having taxonomically the same properties as those of Chlorococcum sp. FERM BP-22262.

19. A method for manufacturing biomass, comprising:
a culture step including the culture method according to claim 1; and
a step of collecting a biofilm on the liquid surface formed through a second culture step.

20. The manufacturing method according to claim 19,
wherein the biomass is oil.


SEQUENCE LISTING (2 sequences)

SEQ ID NO: 1 - DNA, 1129 bp, Botryococcus sudeticus AVFF007
  misc_feature: FERM BP-11420
tacggtcgcttcggtgagtactgctatggccttcctttctacgggggacgggctcctgggattaacttctcgggatccggattccgtgatgatactttgagtaaattggagtgttcaaagcaagcctgcgctctgaacattttagcatggaatatcacgataggactctggcctatcttgttggtctgtaggaccagagtaatgattaagagggacagtcgggggcattcgtatttcattgtcagaggtgaaattcttggatttatgaaagacgaactactgcgaaagcatttgccaaggatgttttcattaatcaagaacgaaagttgggggctcgaagacgattagataccgtcgtagtctcaaccataaacgatgccgactagggattagcagacgtttcattgatgactctgctagcaccttatgtgaaaacaaagtttttgggttccggggggagtatggtcgcaaggctgaaacttaaaggaattgacggaagggcaccaccaggcgtggagcctgcggcttaatttgactcaacacgggaaaacttaccaggtccagacatagtgaggattgacagattgagagctctttcttgattctatgggtggtggtgcatggccgttcttagttggtgggttgccttgtcaggttgattccggtaacgaacgagacctcagctcctaaatagtccatgccgccttttggcggtatgctgacttcttagagggactattggcgttctagccaatggaagtatgaggcaataacaggtctgtgatgcccttagatgttctgggccgcacgcgcgctacactgatgcgttcaacgagcctatccttgaccgagaggtccgggtaatctttgaaaccgcatcgtgatggggctagactattgcaattattagtctttaacgaggaatgcctagtaagcgcgattcatcagatcgcgttgattacgtccctggtacacaccgcccgtcgctcctaccgattgggtgtgctggtgaagtgttcggatcggcaaagctgggggcaactcttgcattgccgagaagttcattaaaccctcccacctagaggaaggagaagtcgtaacaaggtttccgtaggtgaacctgcggaag

SEQ ID NO: 2 - DNA, 1650 bp, Chlorococcum sp. FFG039
  misc_feature: FERM BP-22262
ctgcttatacggtgaaactgcgaatggctcattaaatcagttatagtttatttgatggtacctcctacttggataaccgtaggaaatctagagctaatacatgcgtaaatcccgacttctggaagggacgtatttattagataaaaggccagccgggcttgcccgaccttaggcgaatcatgataacttcacgaatcgcatggccttgtgccggcgatgtttcattcaaatttctgccctatcaactttcgatggtaggatagaggcctaccatggtggtaacgggtgacggaggattagggttcgattccggagagggagcctgagaaacggctaccacatccaaggaaggcagcaggcgcgcaaattacccaatcccgacacggggaggtagtgacaataaataacaatactgggcatttatgtctggtaattggaatgagtacaatgtaaatatcttaacgagtatccattggagggcaagtctggtgccagcagccgcggtaattccagctccaatagcgtatatttaagttgttgcagttaaaaagctcgtagttggatttcgggtgcgtcgacgcggtctgcctctggtatgtactgcgctcggtgcacctttcagctgaatacgctctcctgggcttaactgcttgggaagtggagtcagcagagtgaccttgagcaaacaagagtgttcaaagcaagccgacgctctgaattttttagcatggaatcacacgataggactctggcctatcttgttggtctgtaggaccggagtaatgattaagagggacagtcgggggcattcgtatttcattgtcagaggtgaaattcttggatttatgaaagacgaacttctgcgaaagcatttgccaaggatgttttcattgatcaagaacgaaagttgggggctcgaagacgattagataccgtcgtagtctcaaccataaacgatgccgactagggattggcaggtgttccattgatgaccctgccagcaccttatgagaaatcaaagtttttgggttccggggggagtatggtcgcaaggctgaaacttaaaggaattgacggaagggcaccaccaggcgtggagcctgcggcttaatttgactcaacacggggaaacttaccaggtccagacacggggaggattgacagattgagagctctttcttgattctgtgggtggtggtgcatggccgttcttagttggtgggttgccttgtcaggttgattccggtaacgaacgagacctcagcctgctaaatagtcacgggtaccttggtacacgcctgacttcttagagggactattggcgtctagtcaatggaagtgtgaggcaataacaggtctgtgatgcccttagatgttctgggccgcacgcgcgctacactgatgcattcaacgagcctatccttggccgagaggtccgggtaatctttgaaactgcatcgtgatggggatagattattgcaattattagtcttcaacgaggaatgcctagtaagcgcaagtcatcagcttgcgttgattacgtccctgccctttgtacacaccgcccgtcgctcctaccgattgggtgtgctggtgaagtgttcggattggctccagggatgggcaaccactcctgttgctgagaagaac